(19) United States
(12) Patent Application Publication
(10) Pub. No.: US 2016/0271274 A1
(43) Pub. Date: Sep. 22, 2016

(54) SYNTHESIS AND USE OF TARGETED RADIATION ENHANCING IRON OXIDE-SILICA-GOLD NANOSHELLS FOR IMAGING AND TREATMENT OF CANCER

(71) Applicant: THE JOHNS HOPKINS UNIVERSITY, Baltimore, MD (US)

(72) Inventors: ROBERT IVKOV, ELLICOTT CITY, MD (US); LAUREN WOODARD, ELLLICOTT CITY, MD (US); MARTIN G. POMPER, BALTIMORE, MD (US)

(73) Assignee: THE JOHNS HOPKINS UNIVERSITY, BALTIMORE, MD (US)

(21) Appl. No.: 15/035,012

(22) PCT Filed: Nov. 7, 2014
(86) PCT No.: PCT/US14/64587

§ 371 Date: May 6, 2016

Related U.S. Application Data
(60) Provisional application No. 61/901,209, filed on Nov. 7, 2013.

(51) Int. Cl. A61K 49/18; A61B 5/00; A61B 6/03; A61K 49/08; A61K 9/50; A61K 9/51; A61K 41/00; A61K 49/04; A61B 5/055; A61N 5/10

(52) U.S. Cl. CPC A61K 49/183; A61B 5/055; A61B 5/0066; A61B 6/032; A61N 5/10; A61K 9/5094; A61K 9/5192; A61K 9/5115; A61K 41/0052; A61K 49/0428; A61K 49/08

(57) ABSTRACT

Magnetic iron oxide nanoparticles (MIONs) having silica (SiMION) and gold-silica (AuSiMION) nanoshells, methods of their preparation, and their use in cancer imaging and therapy applications are disclosed.

### FEDERALLY SPONSORED RESEARCH OR DEVELOPMENT

This invention was made with government support under T32-CA130840 awarded by the National Institutes of Health (NIH), Centers of Cancer Nanotechnology Excellence (CCNE) grant number U54CA151838 awarded by the NIH, and agreement DMR-0944772 awarded by the National Science Foundation (NSF). The government has certain rights in the invention.

### BACKGROUND

Most cancer deaths are caused by recurrent and widely disseminated (e.g., metastatic) disease. Late-stage recurrences are typically systemic conditions that are refractory to standard of care therapies. Further, locally-advanced primary or recurrent cancers of the pancreas, head and neck, brain, and liver are particularly problematic. Image-guided interventions are often the primary treatment option for locally advanced disease, whereas systemically-delivered targeted theranostic agents are a viable alternative for treating recurrent and widely disseminated disease.

Heat is mechanical incoherent energy that broadly affects multiple cell processes and proteins in ways that complement the DNA-damaging effects of radiation and chemotherapies. Heat effectively inhibits DNA-damage repair following radiation therapy, making cancer cells more responsive to therapy. Delivering an effective dose of heat to cancer, however, remains a technical barrier.

Magnetic nanoparticle hyperthermia for cancer therapy is an application of alternating magnetic fields (AMFs) in which magnetic nanoparticle heating depends upon both AMF frequency and amplitude (Jordan, et al., Scientific and Clinical Applications of Magnetic Carriers, 569-595 (1997); Rosensweig, J. Magnetism and Magn. Materials 252, 370-374 (2002); Bordelon, et al., Journal of Applied Physics 109, 12904.1-12904.8 (2011). When a region of tissue in an animal or a patient is subjected to an AMF, non-specific Joule heat is deposited into the tissue due to eddy currents. The total non-specific power deposited is proportional to H²f²r²; where H and f are AMF amplitude and frequency, respectively; and r is the radius of the eddy current path, which is related to the radius of tissue exposed to AMF. For some magnetic iron-oxide nanoparticles (MIONs) the heat generating ability, or specific loss power (SLP) with amplitude is proportional to SLP∝H^{x}f, where x can vary between 0 and 3, depending upon the magnetic anisotropy energy of the MIONP construct and the value of the field amplitude (H) relative to MION saturation magnetization (Mₛ). To minimize excess off-target tissue heating while maintaining sufficient energy deposition from MION heating, lower AMF frequencies in the range of 100 kHz to 400 kHz are typically used in mNPH applications (Atkinson, et al., IEEE Trans. Biomed. Eng. 31, 70-75 (1984)). For mNPH to be effective, the magnetic anisotropy energy of the MIONs should be sufficient to enable them to generate higher heating at low field amplitude, or H-values.

Despite the great promise, magnetic nanoparticle hyperthermia (mNHP) has had limited success in clinical applications. This limited success is due, in part, to technical difficulties of selective heat delivery to the target tissue without overheating adjacent normal tissue. For a given magnetic nanoparticle formulation localized in tissue, the amount of heat deposited during mNHP depends on both the intratumoral MION concentration and AMF parameters. Generally, the objective is to develop nanoparticle and AMF device combinations that produce a maximum particle-associated heating rate, or loss power for a given peak amplitude of magnetic field. For many magnetic materials, the loss power increases both with increasing AMF frequency and amplitude, thus motivating development of particles that generate therapeutic heating with safe AMF exposure.

### SUMMARY

In some aspects, the presently disclosed subject matter provides a process for preparing one or more magnetic metal oxide particles having a silica or gold-silica nanoshell, the process comprising: (a) providing a salt solution of a metal; (b) contacting the salt solution of the metal with a precipitant solution to form a reactant solution; (c) rapidly micro-mixing the reactant solution to initiate formation of metal oxide crystals under controlled nucleation conditions; (d) continuing to rapidly micro-mix the reactant solution under high gravity conditions to control crystal growth of one or more metal oxide particles formed therein; (e) optionally coating the one or more metal oxide particles with a surfactant; (f) separating the one or more metal oxide particles from the reactant solution and one or more by-products, if present, formed therein; (g) exposing the one or more coated metal oxide particles to high temperature and high pressure in an inert gas environment for a period of time to form one or more magnetic metal oxide particles; and (h) coating the one or more magnetic metal oxide particles with silica to form one or more magnetic metal oxide particles having a silica nanoshell.

In particular aspects, the process further comprises: (i) amino-terminating the silica coating of the one or more magnetic metal oxide particles having a silica nanoshell; (j) gold seeding the amino-terminated silica coating of the one or more magnetic metal oxide particles having a silica nanoshell; and (k) gold plating the gold-seeded one or more magnetic metal oxide particles having a silica nanoshell to form one or more magnetic metal oxide particles having a gold-silica nanoshell.

In other aspects, the presently disclosed subject matter provides one or more magnetic metal oxide particles having a silica or gold-silica nanoshell prepared by the presently disclosed methods.

In more particular aspects, the presently disclosed subject matter provides a magnetic metal oxide nanoparticle prepared from a high-gravity controlled precipitation reaction, the nanoparticle comprising: (a) iron oxide crystals having a dimension ranging from about 5 nm to about 100 nm; (b) optionally a surfactant coating; and (c) a silica coating; wherein the nanoparticle has a heating property of greater than about 60 W/g Fe in an alternating current (AC) magnetic field having a frequency of ranging from about 50 kHz and to about 1 MHz and an amplitude ranging from about 0.080 kA/m to about 80 kA/m. In even more particular aspects, the magnetic metal oxide nanoparticle further comprises a gold coating.

In yet other aspects, the presently disclosed subject matter provides a biocompatible suspension comprising a magnetic metal oxide nanoparticle having a silica coating or a gold-silica coating prepared by a high-gravity controlled precipitation reaction and water.

In further aspects, the presently disclosed subject matter provides a method for treating a diseased tissue, the method comprising: (a) administering to a tissue or a subject in need of treatment thereof, a therapeutically effective amount of a magnetic nanoparticle having a silica or a gold-silica nanoshell, wherein the nanoparticle comprises iron oxide crystals prepared from a high-gravity controlled precipitation process; (b) subjecting the tissue or subject, or a portion of the tissue or subject to an alternating current (AC) magnetic field having frequency ranging from about 50 kHz to about 1 MHz and having an amplitude (peak-to-peak) ranging from about 0.080 kA/m to about 50 kA/m. In particular aspects, the diseased tissue comprises a cancer tissue.

In some aspects, the presently disclosed subject matter provides a method of imaging a diseased tissue, the method comprising: (a) administering to a tissue or a subject in need of treatment thereof, a therapeutically effective amount of a magnetic nanoparticle having a silica or a gold-silica nanoshell, wherein the magnetic nanoparticle comprises iron oxide crystals prepared from a high-gravity controlled precipitation process; and (b) imaging the magnetic nanoparticle having a silica or a gold-silica nanoshell. In particular embodiments, the imaging is conducted by an imaging technique selected from the group consisting of magnetic resonance imaging, plasmon resonance imaging, x-ray imaging, optical coherence tomography (OCT), and x-ray computed tomography.

In yet further aspects, the presently disclosed subject matter provides a magnetic nanoparticle comprising: (a) a magnetic core comprising an aggregate of at least two magnetic crystalline grains, wherein the aggregate exhibits a collective magnetic phase such that the core has an apparently single magnetic domain phase; (b) a second magnetic phase or magnetic oxide phase differing from the collective or single domain phase of the core, wherein the second magnetic phase or magnetic oxide phase can intercalate and surround the core; wherein at least one magnetic phase exhibits a high-coercive behavior in a magnetic field and at least one other phase exhibits a low-coercive behavior in a magnetic field relative to the high-coercive magnetic phase; (c) optionally a coating; and (d) a silica coating or a gold-silica coating. In particular aspects, the core substantially comprises Fe₃O₄ and the second magnetic phase or magnetic oxide phase substantially comprises γ-Fe₂O₃.

Certain aspects of the presently disclosed subject matter having been stated hereinabove, which are addressed in whole or in part by the presently disclosed subject matter, other aspects will become evident as the description proceeds when taken in connection with the accompanying Examples and Figures as best described herein below.

### BRIEF DESCRIPTION OF THE FIGURES

Having thus described the presently disclosed subject matter in general terms, reference will now be made to the accompanying Figures, which are not necessarily drawn to scale, and wherein:

FIG. 1 is a representative synthesis schematic of gold-silica-coated MIONs. Iron oxide cores (MIONs, 1) were coated with silica using tetraethylorthosilicate to form Si-MIONs (2). The Si-MIONs were amino-terminated using 3-aminopropyltrimethoxysilane and seeded by a colloidal gold solution containing 1-2 nm gold seeds. Finally a gold shell was grown on the surface by the reduction of chloroauric acid to form AuSi-MIONs (3);

FIG. 2 is a graphical representation of the particle diameter of the presently disclosed nanoparticles obtained by dynamic light scattering (DLS), wherein the DLS curves increase as the silica coating thickens based on equivalences of TEOS added during synthesis (top spectrum); on bottom, size increases from citrate-stabilized MIONs (55 nm) to silica-coated MIONs (75 nm) and finally to the gold/silica-coated MIONs (130 nm);

FIGS. 3A-C are TEMs of (A) citrate-stabilized MION particles (uncoated MIONs), (B) SiMIONs (SiMIONs), and (C) AuSiMIONs (AuSiMIONs);

FIG. 4 shows the specific power loss versus field strength curves comparing heating efficiency of uncoated MIONs to SiMIONs and AuSiMIONs. Heat output is measured as a function of AMF amplitude at a frequency of 150 kHz;

FIG. 5 shows AMF hyperthermia treatment of tumors with MIONs and AuSiMIONs at 500 Oe and 150 kHz. The field was turned on at 30 s and treatment lasted for 1200 s. Figure has been normalized and compares the change in intratumoral temperature during AMF hyperthermia using MIONs and AuSiMIONs;

FIG. 6 shows the magnetic characterization of iron oxide-based nanoparticles both as a function of coating type and coating thickness. Measurements were performed at 5K (both zero field cooled and field cooled) and at 300K. Only SiMIONs with average diameters of 67 nm and 78 nm displayed magnetic loop shifts;

FIG. 7 shows mice with prostate cancer tumors in the right hind legs that were imaged with X-ray CT both before and after the injection of presently disclosed nanoparticles. Only the AuSiMIONs improved image contrast so that the tumor could be visualized;

FIGS. 8a-8e illustrate the physical characterization of representative presently disclosed MIONs: (a) Dynamic light scattering (DLS) of (1) MIONs—55 nm, (2) Si-MIONs—75 nm and (3) AuSi-MIONs—140 nm; (b) SQUID magnetometry measurements of magnetization of MIONs as a function of external field strength. Data are normalized by solid content, reducing measured magnetic contribution; (c) Transmission electron microscopy (TEM) of (1) MION cores, (2) silica-coated MIONs and (3) gold and silica-coated MIONs; scale bars are 100 nm; (d) Small angle neutron scattering (SANS) data and analysis of MION size and shape; and (e) Dimensions and 3D models of MIONs based on SANS analysis;

FIGS. 9a-9f demonstrate the theranostic potential of the presently disclosed AuSi-MIONs: (a) Photograph showing AuSi-MIONs drawn by permanent magnets demonstrating potential for magnetic vectorization; (b) Illustration of potential for magnetic vectorization; and (c) MR imaging contrast of MIONs (1), Si-MIONs (2) and AuSi-MIONs (3). Imaging of gel phantoms over a range of 0-80 μg/mL (0-1.4 mM) based on iron content, showing T₂ effect as iron concentration increases (top). T₂ relaxation (ms) calculated from spin-echo MR imaging of phantoms (bottom). Inset shows concentration (mM) versus 1/T₂, the slope of which gives transverse relaxivity (R₂) in units of mM⁻¹s⁻¹; (d) Signal intensity from MION phantoms over a range of 0-7 mg/mL (based on iron content) demonstrating CT contrast with gold (top). CT contrast, measured in Hounsfield units (HU), was calculated for each sample and were plotted versus iron concentration (bottom); (e) Specific loss power (SLP), a measure of heating efficiency in an alternating magnetic field, was measured for MIONs (square), Si-MIONs (diamond) and AuSi-MIONs (triangle) at a frequency of 150 kHz±5 kHz over a range of amplitudes from 10 to 80 kA/m; and (f) Comparison of laser-induced heating, reported as specific absorption rates (SARs, normalized by iron content) between MIONs and AuSi-MIONs. A 5.5 W laparoscopic laser was centered on each solution for 15 seconds. The change in temperature was monitored and SARs were calculated for each sample;

FIGS. 10a-10c show the in vivo evaluation of AuSiMIONs: (a) In vivo CT imaging in nude male mice bearing human prostate (LAPC-4) cancer xenograft tumours. Control: saline only injection (left)—the red oval denotes the location of the tumour, which is invisible without added contrast. MIONs (middle): injection concentration of 5.5 mg Fe/cm³ tumour. Iron oxide demonstrates insufficient x-ray contrast with CT rendering the tumour invisible. AuSi-MIONs (right): injection concentration of 5.5 mg Fe/cm³ tumour. The AuSi-MIONs are visible in the tumour indicated by increased signal; (b) following CT imaging, mice were placed in an AMF device (150 kHz, 40 kA/m) and an approximately 6° C. rise of tumour temperature was measured with RF-resistant optical fiber temperature probe inserted into tumours loaded with either MIONs or AuSi-MIONs; and (c) mice were euthanized and tumour tissues were collected for staining 72 h post AMF exposure (Row I: H&E, row II: Prussian blue and row III: silver enhancement stain). The control shows no iron oxide or gold present. Tissues from the mouse injected with MIONs show iron oxide particles in the H&E stain, iron staining (blue) with Prussian blue and no response to the silver enhancement stain. Tissues from the mouse injected with AuSi-MIONs show a dark purple color from the gold nanoparticles in the H&E stain and iron staining (blue) with Prussian blue. Dark black staining of the gold from the silver enhancement stain, which only stains metallic gold or silver, can be seen in column III. Whole tumor images are composites created from separate 4× images; magnified images were obtained at 20×;

FIG. 11 shows (top) attempts to fit SANS data using single models. MIONs (left), Si-MIONs (middle) and AuSi-MIONs (right); (bottom) individual fits using best fit numbers obtained from the summation of two models (stacked discs+triaxialellipsoid for MIONs and core-shell+triaxialellipsoid for Si-MIONs and AuSi-MIONs); and

FIG. 12 shows the CT monitoring of AuSi-MION location in LAPC-4 model (red oval denotes tumour location). Following intratumoral injection of AuSi-MIONs (5.5 mg iron oxide per cm³ of tumor) into the hind leg of a nude mouse, signal intensity was monitored over 13 days. CT scans were performed immediately following the injection of particles, on day 6 and on day 13. Particles were still clearly visible on day 13 with no decrease in signal intensity.

### DETAILED DESCRIPTION

The presently disclosed subject matter now will be described more fully hereinafter with reference to the accompanying Figures, in which some, but not all embodiments of the inventions are shown. Like numbers refer to like elements throughout. The presently disclosed subject matter may be embodied in many different forms and should not be construed as limited to the embodiments set forth herein; rather, these embodiments are provided so that this disclosure will satisfy applicable legal requirements. Indeed, many modifications and other embodiments of the presently disclosed subject matter set forth herein will come to mind to one skilled in the art to which the presently disclosed subject matter pertains having the benefit of the teachings presented in the foregoing descriptions and the associated Figures. Therefore, it is to be understood that the presently disclosed subject matter is not to be limited to the specific embodiments disclosed and that modifications and other embodiments are intended to be included within the scope of the appended claims.

Magnetic iron oxide nanoparticles (MIONs) are used for cancer therapy because they can generate heat via hysteresis in alternating magnetic fields. MIONs can provide localized, cell-specific and intense heating when exposed to alternating magnetic fields (AMFs), but only when prepared with appropriate magnetic anisotropy. Dennis, et al., Nanotechnology 2009, 20 (39); Dennis and Ivkov, Int. J. Hyperthermia 2013, 29 (8), 715-729.

There is some debate, however, regarding the potential limitations of MIONs to treat micrometastases or small cell clusters. Hedayati, et al., Nanomedicine 2013, 8 (1), 29-41. Further, uncoated MIONs are insufficiently biocompatible for systemic delivery and may even be toxic. MIONs require biocompatible coatings that also offer surfaces for binding cell-specific ligands. To enhance biocompatibility, MIONs are typically coated with polymers, such as dextran, thereby producing a “soft shell.”

Alternatively, gold is attractive as a coating material because its biocompatibility has been demonstrated in human clinical trials of gold nanoparticles. It also provides a convenient surface for chemical conjugation of anti-cancer agents via thiol (—SH) group linkage. Gold, or gold-coated nanoparticles, also displays optical responsive properties via plasmon resonance to provide optical imaging or heating depending upon the wavelength of the incident light. By adjusting the gold shell thickness, one can tune the particle to absorb light at near-IR wavelengths, thereby increasing local temperature and inducing cell death. Gobin, et al., Nano Letters 2007, 7 (7), 1929-1934; Huang, et al., Lasers in Medical Science 2008, 23 (3), 217-228; Lal, et al., Accounts of Chemical Research 2008, 41 (12), 1842-1851; and O'Neal, et al., Cancer Letters 2004, 209 (2), 171-176.

Finally, gold can provide x-ray contrast to enhance x-ray imaging, and it is a known radiation enhancing material when exposed to x-rays (production of photo or Auger electrons). Lechtman, et al., Physics in Medicine and Biology 2011, 56 (15), 4631-4647. Thus, a gold coating provides a novel solution to the two challenges facing MIONs—it enhances the imaging and therapeutic (theranostic) potential by enhancing radiation and heating potential, adding optical and x-ray contrast capability, and it coats the MIONs with a biocompatible surface that facilitates functionalization for targeting. A gold layer also adds additional optical imaging capability that may enhance utility via optical coherence tomography (OCT). Oldenburg, et al., Optics Express 2006, 14 (15), 6724-6738. Addition of gold coating to magnetic nanoparticles offers new promise to enhance therapy via hyperthermia and radiation therapy by combining MION-based heat delivery with laser heating, radiation enhancement properties of gold, and multifunctional imaging (magnetic resonance, OCT, and x-ray CT).

Accordingly, the presently disclosed subject matter describes the synthesis and subsequent use of iron oxide-silica-gold nanoshells for imaging and treatment of cancer. Heat, a potent anti-cancer agent, also is known to be a radiosensitizer. Magnetic iron oxide nanoparticles are responsive to magnetic fields and thus are inherently MRI contrast agents. When such nanoparticles have appropriate anisotropy, they produce significant heat when placed in an alternating magnetic field that can be used for cancer hyperthermia. Coating these particles with gold introduces optical responsiveness (i.e., plasmon resonance) for both imaging and heating, x-ray opacity for enhanced x-ray contrast and radiation therapy, and reduced toxicity. Further, targeting moieties can be added to the gold surface via thiol (—SH) chemistry, enabling cell-specific localization of the nanoparticles, thereby reducing damage to the surrounding normal tissues. The presently disclosed silica-gold coating process preserves the magnetic properties of the iron oxide nanoparticle platform thereby enabling tri-modality imaging and therapeutic potential.

The presently disclosed subject matter provides a multifunctional imaging and therapy nanoparticle platform by coating a MION with silica or gold-silica nanoshells. Such coatings provide a “hard shell” and add optical responsiveness (i.e., plasmon resonance) to the magnetic properties of the particles.

The presently disclosed nanoparticles were characterized by DLS, TEM, and SQUID. Magnetic characterization with SQUID magnetometry produced hysteresis loops that were symmetrical about zero for uncoated MIONs. Some SiMIONs, however, displayed a distinct loop shift. The presently disclosed AuSiMIONs can be used in cancer imaging and therapy applications.

Further, the presently disclosed subject matter addresses a critical unmet need in radiation oncology for treatment of locally advanced and disseminated cancers by offering an innovative, minimally invasive, image-guided therapeutic tool. Gold-coated magnetic nanoparticles offer new promise to enhance therapy via hyperthermia and radiation therapy by combining MION-based heat delivery with laser heating, radiation enhancement properties of gold, and multifunctional imaging (magnetic resonance, OCT, and x-ray CT). Quantitative nuclear imaging will enable individualized image guided treatment planning and dosimetry. In addition, functionalization of the particles with various targeting moieties (i. e., RGD peptides for integrin targeting in glioblastoma multiforme) will allow cell-specific localization of the nanoparticles. The ability to target these nanoparticles to cancer cells will result in minimal damage to surrounding normal tissues following subsequent hyperthermia and radiation therapies.

As provided herein, the syntheses of SiMIONs and AuSiMIONs were confirmed by transmission electron microscopy (TEM) and dynamic light scattering (DLS). The heating efficiencies of the presently disclosed coated MIONs decreased slightly, which, without wishing to be bound to any one theory, it is thought that the decrease in heating efficiency may be due to diamagnetic shielding. Further, SQUID magnetometry of 67 nm and 78 nm SiMIONs displayed a magnetic loop shift, which, again without wishing to be bound to any one theory, is thought to be due to pinned/uncompensated spins rather than an exchange bias. The presently disclosed AuSiMION nanoparticles were successfully used as a CT contrast enhancer in vivo and to efficiently heat a subcutaneous tumor in vivo when introduced to an alternating magnetic field.

### I. METHODS FOR MAKING IRON OXIDE NANOPARTICLES HAVING SILICA OR GOLD-SILICA NANOSHELLS

In some embodiments, the presently disclosed subject matter provides a process for preparing one or more magnetic metal oxide particles having a silica or gold-silica nanoshell, the process comprising: (a) providing a salt solution of a metal; (b) contacting the salt solution of the metal with a precipitant solution to form a reactant solution; (c) rapidly micro-mixing the reactant solution to initiate formation of metal oxide crystals under controlled nucleation conditions; (d) continuing to rapidly micro-mix the reactant solution under high gravity conditions to control crystal growth of one or more metal oxide particles formed therein; (e) optionally coating the one or more metal oxide particles with a surfactant; (f) separating the one or more metal oxide particles from the reactant solution and one or more by-products, if present, formed therein; (g) exposing the one or more coated metal oxide particles to high temperature and high pressure in an inert gas environment for a period of time to form one or more magnetic metal oxide particles; and (h) coating the one or more magnetic metal oxide particles with silica to form one or more magnetic metal oxide particles having a silica nanoshell.

In particular embodiments, the process further comprises: (i) amino-terminating the silica coating of the one or more magnetic metal oxide particles having a silica nanoshell; (j) gold seeding the amino-terminated silica coating of the one or more magnetic metal oxide particles having a silica nanoshell; and (k) gold plating the gold-seeded one or more magnetic metal oxide particles having a silica nanoshell to form one or more magnetic metal oxide particles having a gold-silica nanoshell.

In yet further embodiments, the process further comprises coating the one or more magnetic metal oxide particles having a gold-silica nanoshell with a biocompatible coating. In particular embodiments, the process further comprises binding a ligand to the biocompatible coating.

In some embodiments, the reactant solution comprises an iron precursor solution comprising anhydrous FeCl₃ and FeCl₂.4H₂O in hydrochloric acid. In some embodiments, the salt solution comprises a metal salt comprising a metal selected from the group consisting of Fe, Co, Ni, and Sm. In further embodiments, the metal salt comprises an anionic species selected from the group consisting of chloride, bromide, fluoride, iodide, nitrate (NO₃), sulfate (SO₄), chlorate (ClO₄), and phosphate (PO₄).

In some embodiments, the precipitant solution comprises ammonia. In other embodiments, the precipitant solution comprises at least one member selected from the group consisting of NaOH, ammonium hydroxide (NH₄OH), and another hydroxide of Group I or II elements from the Periodic Table of elements. In further embodiments, the reactant solution comprises at least one member selected from the group consisting of a hydroxide, a carbonate, and a phosphate.

In particular embodiments, the exposing of the one or more metal oxide particles to high temperature and high pressure is conducted at about 130° C. for about 5 hours.

In further embodiments, as described in more detail herein below, the presently disclosed subject matter provides one or more magnetic metal oxide particles having a silica or gold-silica nanoshell prepared by the presently disclosed methods.

One characteristic of the nano- or micro-particles produced by these methods is that they need to provide uniform heating at many sites. Such uniform heating requires a predictable or uniform dose and dosimetry. The alternating magnetic field (AMF) amplitude must be uniformly applied to a large volume of tissue. The appreciable tissue volume exposure limits field amplitude to about 15-24 kA/m. Therefore, the presently disclosed particles are capable of producing substantial heating at low amplitude fields. To provide these characteristics, the presently disclosed subject matter provides high-gravity controlled precipitation methods to prepare the base iron oxide crystal. The iron oxide crystals are coated with a weak, organic acid, such as citric acid, to ensure charge stabilization, resulting in colloid stability.

High Gravity Controlled Precipitation (HGCP) Technology

Nano- or micro-particles can be obtained by rapid micro-mixing of reactants to enhance nucleation while suppressing crystal growth. Thorough micro-mixing leads to uniform crystal growth and therefore uniform particle size can be obtained. On the other hand, insufficient micro-mixing will lead to growth disparity among different nuclei, resulting in a wide particle size distribution (PSD). There are two characteristic time parameters in crystallization: the induction time (T) and the micro-mixing time (tₘ). When tₘ<<T, the nucleation rate will be nearly uniform spatially, and the PSD can be controlled at a uniform level. This can be achieved by a High Gravity Controlled Precipitation (HGCP) reactor which utilizes a rotating packed bed to intensify mass and heat transfer in multiphase systems. During rotation, the fluids going through the packed bed are spread and split into thin films, threads and very fine droplets under the high shear force created by the high gravity. This results in intense micro-mixing between the fluid elements by 1-3 orders of magnitude. The micro-mixing time (tₘ) in this process is estimated to be around the magnitude of the order of 10-100 μs in the presently disclosed methods.

### II. COMPOSITIONS COMPRISING IRON OXIDE NANOPARTICLES HAVING A SILICA OR GOLD-SILICA NANOSHELL

As used herein, the terms “nanoparticle” refers to one or more structures that have at least one dimension, e.g., a height, width, length, and/or depth, in a range from about one nanometer (nm), i.e., 1×10⁻⁹ meters, to about 999 nm, including any integer value, and fractional values thereof, including about 1, 2, 5, 10, 20, 30, 40, 50, 60, 70, 80, 90, 100, 200, 500, 600, 700, 800, 900, 999 nm and the like.

In some embodiments, the presently disclosed subject matter provides a magnetic metal oxide nanoparticle prepared from a high-gravity controlled precipitation reaction, the nanoparticle comprising: (a) iron oxide crystals having a dimension ranging from about 5 nm to about 100 nm; (b) optionally a surfactant coating; and (c) a silica coating; wherein the nanoparticle has a heating property of greater than about 60 W/g Fe in an alternating current (AC) magnetic field having a frequency of ranging from about 50 kHz and to about 1 MHz and an amplitude ranging from about 0.080 kA/m to about 80 kA/m.

In particular embodiments, the magnetic metal oxide nanoparticle further comprises a gold coating. In yet more particular embodiments, the gold-coated magnetic metal oxide nanoparticle further comprising a biocompatible coating. In yet even more particular embodiments, the gold-coated magnetic metal oxide nanoparticle comprising a biocompatible coating further comprises a ligand.

Generally, the one or more magnetic metal oxide particles have a substantially isotropic shape and have a dimension ranging from about 50 nm to about 100 nm. More particularly, the particles comprise about 76% Fe₃O₄ and about 24% γ-Fe₂O₃ and are substantially free of Fe(OH)₂.

In further embodiments, the presently disclosed subject matter provides a magnetic nanoparticle comprising: (a) a magnetic core comprising an aggregate of at least two magnetic crystalline grains, wherein the aggregate exhibits a collective magnetic phase such that the core has an apparently single magnetic domain phase; (b) a second magnetic phase or magnetic oxide phase differing from the collective or single domain phase of the core, wherein the second magnetic phase or magnetic oxide phase can intercalate and surround the core; wherein at least one magnetic phase exhibits a high-coercive behavior in a magnetic field and at least one other phase exhibits a low-coercive behavior in a magnetic field relative to the high-coercive magnetic phase; (c) optionally a surfactant coating; and (d) a silica or a gold-silica coating. More particularly, the core substantially comprises Fe₃O₄ and the second magnetic phase or magnetic oxide phase substantially comprises γ-Fe₂O₃.

In some embodiments, the nanoparticles may further comprise an external coating. The coating may enhance the heating properties of the nanoparticles and/or may comprise radioactive or potentially radioactive elements. Suitable materials for the coating include synthetic and biological polymers, copolymers and polymer blends, and inorganic materials. Polymer materials may include various combinations of polymers of acrylates, siloxanes, styrenes, acetates, akylene glycols, such as polyethylene glycol, alkylenes, alkylene oxides, parylenes, lactic acid, and glycolic acid. Further suitable coating materials include a hydrogel polymer, a histidine-containing polymer, and a combination of a hydrogel polymer and a histidine-containing polymer.

Coating materials may also include combinations of biological materials, such as a polysaccharide, a polyaminoacid, a protein, a lipid, a glycerol, and a fatty acid. Examples of other biological materials suitable for use herein include heparin, heparin sulfate, chondroitin sulfate, chitin, chitosan, cellulose, dextran, alginate, starch, carbohydrate, and glycosaminoglycan. Examples of proteins useful herein include an extracellular matrix protein, proteoglycan, glycoprotein, albumin, peptide, and gelatin. These materials may also be used in combination with any suitable synthetic polymer material.

Inorganic coating materials may include any combination of a metal, a metal alloy, and a ceramic. Examples of ceramic materials suitable for use herein include a hydroxyapatite, silicon carbide, carboxylate, sulfonate, phosphate, ferrite, phosphonate, and oxides of Group IV elements of the Periodic Table of Elements. These materials may form a composite coating that may also contain one or more biological or synthetic polymers. Where the magnetic particle is formed from a magnetic material that is biocompatible, the surface of the particle itself operates as the biocompatible coating.

The coating material may also serve to facilitate transport of the nanoparticles into a cell, a process known as transfection. Such coating materials, referred to as transfection agents, include vectors, prions, polyaminoacids, cationic liposomes, amphiphiles, and non-liposomal lipids or any combination thereof. A suitable vector may be a plasmid, a virus, a phage, a viron, a viral coat. The nanoprobe coating may be a composite of any combination of transfection agent with organic and inorganic materials, such that the particular combination may be tailored for a particular type of a diseased material and a specific location within a patient's body.

In further embodiments, the presently disclosed subject matter provides a biocompatible suspension comprising a presently disclosed magnetic metal oxide nanoparticle and water.

In still further embodiments, the presently disclosed subject matter provides a kit for preventing and/or treating a cell disorder or diseased tissue by using at least one magnetic metal oxide particle of the presently disclosed subject matter. In an embodiment, the presently disclosed subject matter provides a kit for treating a diseased tissue, the kit comprising a magnetic metal oxide nanoparticle prepared from a high-gravity controlled precipitation reaction.

### III. METHODS FOR USING IRON OXIDE NANOPARTICLES HAVING A SILICA OR GOLD-SILICA NANOSHELL

A. Methods for Treating a Diseased Tissue

Metastatic cancer is characterized by diffuse disease with occult and widespread metastatic lesions, and is typically refractory to standard of care therapies. Heat is a potent sensitizer of cancer to both radiation and some chemotherapeutic agents. However, delivering the heat selectively to cancer tumors, particularly those typical of metastatic disease represents a challenge that has not yet been adequately addressed. Magnetic nanoparticles that are capable of localizing to these sites and heating when exposed to an AC magnetic field allow depositing of heat to these tumor sites with little adverse damage to surrounding normal tissue. To be effective, the nanoparticles must be capable of generating substantial heat (>100 W/g Fe) when exposed to low frequency (100 kHz to 300 kHz) and low power (peak-to-peak amplitude 10 kA/m to 30 kA/m) AC fields. These latter constraints are necessary to avoid overheating the patient by nonspecific heating that results from interactions of large volumes of tissue with the electromagnetic field.

Generally, in some embodiments, the presently disclosed subject matter provides a method for treating a diseased tissue, the method comprising: (a) administering to a tissue or a subject in need of treatment thereof, a therapeutically effective amount of a magnetic nanoparticle comprising a silica or a gold-silica nanoshell, wherein the nanoparticle comprises iron oxide crystals prepared from a high-gravity controlled precipitation process; (b) subjecting the tissue or subject, or a portion of the tissue or subject to an alternating current (AC) magnetic field having frequency ranging from about 50 kHz to about 1 MHz and having an amplitude (peak-to-peak) ranging from about 0.080 kA/m to about 50 kA/m.

In one embodiment, the presently disclosed nanoparticles are used as therapeutic drugs for cell disorders. In some embodiments, the cell disorder may be, but is not limited to, cancer. In other embodiments, the presently disclosed nanoparticles may be used in other diseases, where eliminating aberrant cells or modulating an aberrant cellular function would be useful. Aberrant cells include, but are not limited to, cells infected by a virus and cells infected by a bacterium. Therefore, the cell disorder may be associated with diseases, such as cancer, diseases of the immune system, pathogen-borne diseases, and undesirable targets, such as toxins, reactions to organ transplants, hormone-related diseases, and non-cancerous diseased cells or tissue.

In some embodiments, the presently disclosed subject matter has use in treating a cell disorder, such as cancer, and thus provides a method of treating a cell disorder. More specifically, in some embodiments, the method has use in treating or preventing a cell disorder in a subject.

The presently disclosed methods generally comprise contacting at least one cell with at least one nanoparticle. The methods thus can be practiced in vitro, in vivo, and ex vivo. They accordingly may be practiced, for example, as a research method to identify compounds or to determine the effects of compounds and concentrations of compounds, as a therapeutic method of treating a cell disorder, and as a method to prevent a cell disorder. In embodiments where the method is a method of treating, it can be a method of therapy (e.g., a therapeutic method) in which the amount administered is an amount that is effective for reducing or eliminating a cell disorder. In embodiments where the method is a method of prevention, the amount is an amount sufficient to prevent the cell disorder from occurring or sufficient to reduce the severity of the cell disorder if it does occur.

A presently disclosed nanoparticle can be targeted to a cell with a disorder by using ligands on the nanoparticle. The ligand may be a polyclonal antibody, a monoclonal antibody, a chimeric antibody, a humanized antibody, a human antibody, a recombinant antibody, a bispecific antibody, an antibody fragment, a recombinant single chain antibody fragment, or any combination of the above.

The choice of a marker (antigen) may be important in the targeted therapy methods of the presently disclosed subject matter. Although not limited thereto, use and selection of markers is most prevalent in cancer immunotherapy. For breast cancer and its metastases, a specific marker or markers may be selected from cell surface markers such as, for example, members of the MUC-type mucin family, an epithelial growth factor (EGFR) receptor, a carcinoembryonic antigen (CEA), a human carcinoma antigen, a vascular endothelial growth factor (VEGF) antigen, a melanoma antigen (MAGE) gene, family antigen, a T/Tn antigen, a hormone receptor, growth factor receptors, a cluster designation/differentiation (CD) antigen, a tumor suppressor gene, a cell cycle regulator, an oncogene, an oncogene receptor, a proliferation marker, an adhesion molecule, a proteinase involved in degradation of extracellular matrix, a malignant transformation related factor, an apoptosis related factor, a human carcinoma antigen, glycoprotein antigens, DF3, 4F2, MGFM antigens, breast tumor antigen CA 15-3, calponin, cathepsin, CD 31 antigen, proliferating cell nuclear antigen 10 (PC 10), and pS2.

For other forms of cancer and their metastases, a specific marker or markers may be selected from cell surface markers such as, for example, a member of vascular endothelial growth factor receptor (VEGFR) family, a member of carcinoembryonic antigen (CEA) family, a type of anti-idiotypic mAB, a type of ganglioside mimic, a member of cluster designation/differentiation antigens, a member of epidermal growth factor receptor (EGFR) family, a type of a cellular adhesion molecule, a member of MUC-type mucin family, a type of cancer antigen (CA), a type of a matrix metalloproteinase, a type of glycoprotein antigen, a type of melanoma associated antigen (MAA), a proteolytic enzyme, a calmodulin, a member of tumor necrosis factor (TNF) receptor family, a type of angiogenesis marker, a melanoma antigen recognized by T cells (MART) antigen, a member of melanoma antigen encoding gene (MAGE) family, a prostate membrane specific antigen (PMSA), a small cell lung carcinoma antigen (SCLCA), a T/Tn antigen, a hormone receptor, a tumor suppressor gene antigen, a cell cycle regulator antigen, an oncogene antigen, an oncogene receptor antigen, a proliferation marker, a proteinase involved in degradation of extracellular matrix, a malignant transformation related factor, an apoptosis-related factor, a type of human carcinoma antigen.

For ovarian cancers and their metastases, a specific marker or markers may be selected from cell surface markers such as, for example, one of ERBB2 (HER-2) antigen and CD64 antigen. For ovarian and/or gastric cancers and their metastases, a specific marker or markers may be selected from cell surface markers such as, for example, a polymorphic epithelial mucin (PEM). For ovarian cancers and their metastases, a specific marker or markers may be selected from cell surface markers such as, for example, one of cancer antigen 125 (CA125) or matrix metalloproteinase 2 (MMP-2). For gastric cancers and their metastases, a specific marker or markers may be selected from cell surface markers such as, for example, one of CA 19-9 antigen and CA242 antigen.

For non small-cell lung cancer (NSCLC), colorectal cancer (CRC) and their metastases, a specific marker or markers may be selected from cell surface markers such as, for example, vascular endothelial growth factor receptor (VEGFR), anti-idiotypic mAb, and carcinoembryonic antigen (CEA) mimic. For at least one of small-cell lung cancer (SCLC), malignant melanoma, and their metastases, a specific marker or markers may be selected from cell surface markers such as, for example, anti-idiotypic mAB or GD3 ganglioside mimic. For melanoma cancers and their metastases, a specific marker or markers may be selected from cell surface markers such as, for example, a melanoma associated antigen (MAA). For small cell lung cancers and their metastases, a specific marker or markers may be selected from cell surface markers such as, for example, a small cell lung carcinoma antigen (SCLCA).

For colorectal cancer (CRC) and/or locally advanced or metastatic head and/or neck cancer, a specific marker or markers may be selected from cell surface markers such as, for example, epidermal growth factor receptor (EGFR). For Duke's colorectal cancer (CRC) and its metastases, a specific marker or markers may be selected from cell surface markers such as, for example, Ep-CAM antigen.

For non-Hodgkin's lymphoma (NHL) and its metastases, a specific marker or markers may be selected from cell surface markers such as, for example, cluster designation/differentiation (CD) 20 antigen or CD22 antigen. For B-cell chronic lymphocytic leukemia and associated metastases, a specific marker or markers may be selected from cell surface markers such as, for example, CD52 antigen. For acute myelogenous leukemia and its metastases, a specific marker or markers may be selected from cell surface markers such as, for example, CD33 antigen.

For prostate cancers and their metastases, a specific marker or markers may be selected from cell surface markers such as, for example, prostate membrane specific antigen (PMSA). For carcinomatous meningitis and their metastases, a specific marker or markers may be selected from cell surface markers such as, for example, one of a vascular endothelial growth factor receptor (VEGFR) or an epithelial associated glycoprotein, for example, HMFGI (human milk fat globulin) antigen.

For lung, ovarian, colon, and melanoma cancers and their metastases, a specific marker or markers may be selected from cell surface markers such as, for example, B7-H1 protein. For colon, breast, lung, stomach, cervix, and uterine cancers and their metastases, a specific marker or markers may be selected from cell surface markers such as, for example, TRAIL Receptor-1 protein, a member of the tumor necrosis factor receptor family of proteins. For ovarian, pancreatic, non-small cell lung, breast, and head and neck cancers and their metastases, a specific marker or markers may be selected from cell surface markers such as, for example, EGFR (epidermal growth factor receptor).

For anti-angiogenesis targeting of tumor blood supply, a specific marker or markers may be selected from cell surface markers such as, for example, Integrin .alpha.v.beta.3, a cell surface marker specific to endothelial cells of growing blood vessels.

For targeting of colon and bladder cancer and their metastases, a specific marker or markers may be selected from cell surface markers such as, for example, RAS, a signaling molecule that transmits signals from the external environment to the nucleus. A mutated form of RAS is found in many cancers.

The cell comprising the target may express several types of markers. One or more nanoparticles may attach to the cell via a ligand. The nanoparticle may be designed such it remains externally on the cell or may be internalized into the cell comprising the target. Once bound to the cell, the magnetic nanoparticle heats in response to the energy absorbed. For example, the magnetic nanoparticle may heat through hysteresis losses in response to an AMF. The heat may pass through the coating or through interstitial regions to the cell, for example via convection, conduction, radiation, or any combination of these heat transfer mechanisms. The heated cell becomes damaged, preferably in a manner that causes irreparable damage. When a sufficient amount of energy is transferred by the nanoparticle to the cell, the cell dies via necrosis, apoptosis or another mechanism.

The nanoparticles may comprise one or more ligands that target and attach to a biological marker. Suitable ligands for use herein include, but are not limited to, proteins, peptides, antibodies, antibody fragments, saccharides, carbohydrates, glycans, cytokines, chemokines, nucleotides, lectins, lipids, receptors, steroids, neurotransmitters, Cluster Designation/Differentiation (CD) markers, and imprinted polymers and the like. The preferred protein ligands include, for example, cell surface proteins, membrane proteins, proteoglycans, glycoproteins, peptides and the like. The preferred nucleotide ligands include, for example, complete nucleotides, complimentary nucleotides, and nucleotide fragments. The preferred lipid ligands include, for example, phospholipids, glycolipids, and the like.

Covalent bonding may be achieved with a linker molecule. Examples of functional groups used in linking reactions include amines, sulfhydryls, carbohydrates, carboxyls, hydroxyls and the like. The linking agent may be a homobifunctional or heterobifunctional crosslinking reagent, for example, carbodiimides, sulfo-NHS esters linkers and the like. The linking agent may also be an aldehyde crosslinking reagent such as glutaraldehyde.

In an embodiment, the ligand may target one or more markers on a cancer cell. In another embodiment, the ligand may target a predetermined target associated with a disease of the patient's immune system. The particular target and one or more ligands may be specific to, but not limited to, the type of the immune disease. The ligand may have an affinity for a cell marker or markers of interest. The marker or markers may be selected such that they represent a viable target on T cells or B cells of the patient's immune system. The ligand may have an affinity for a target associated with a disease of the patient's immune system such as, for example, a protein, a cytokine, a chemokine, an infectious organism, and the like. For rheumatoid arthritis, a specific marker or markers may be selected from cell surface markers such as, for example, one of CD52 antigen, tumor necrosis factor (TNF), and CD25 antigen. For rheumatoid arthritis and/or vasculitis, a specific marker or markers may be selected from cell surface markers such as, for example, CD4 antigen. For vasculitis, a specific marker or markers may be selected from cell surface markers such as, for example, CD18 antigen. For multiple sclerosis, a specific marker or markers may be selected from cell surface markers such as, for example, CD52 antigen.

In still another embodiment, the ligand targets a predetermined target associated with a pathogen-borne condition. The particular target and ligand may be specific to, but not limited to, the type of the pathogen-borne condition. A pathogen is defined as any disease-producing agent such as, for example, a bacterium, a virus, a microorganism, a fungus, and a parasite. For a pathogen-borne condition, the ligand for therapy utilizing nanoparticles may be selected to target the pathogen itself. For a bacterial condition, a predetermined target may be the bacteria itself, for example, one of Escherichia coli or Bacillus anthracis. For a viral condition, a predetermined target may be the virus itself, for example, one of Cytomegalovirus (CMV), Epstein-Barr virus (EBV), a hepatitis virus, such as Hepatitis B virus, human immunodeficiency virus, such as HIV, HIV-1, or HIV-2, or a herpes virus, such as Herpes virus 6. For a parasitic condition, a predetermined target may be the parasite itself, for example, one of Trypanasoma cruzi, Kinetoplastid, Schistosoma mansoni, Schistosoma japonicum or Schistosoma brucei. For a fungal condition, a predetermined target may be the fungus itself, for example, one of Aspergillus, Cryptococcus neoformans or Rhizomucor.

In another embodiment, the ligand targets a predetermined target associated with an undesirable target material. The particular target and ligand may be specific to, but not limited to, the type of the undesirable target. An undesirable target is a target that may be an undesirable material. Undesirable material is material associated with a disease or an undesirable condition, but which may also be present in a normal condition. For example, the undesirable material may be present at elevated concentrations or otherwise be altered in the disease or undesirable state. The ligand may have an affinity for the undesirable target or for biological molecular pathways related to the undesirable target. The ligand may have an affinity for a cell marker or markers associated with the undesirable target material. For arteriosclerosis, a predetermined target may be, for example, apolipoprotein B on low density lipoprotein (LDL). An undesirable material may be adipose tissue or cellulite for obesity, associated with obesity, or a precursor to obesity. A predetermined marker or markers for obesity maybe selected from cell surface markers such as, for example, one of gastric inhibitory polypeptide receptor and CD36 antigen. Another undesirable predetermined target may be clotted blood.

In another embodiment, the ligand targets a predetermined target associated with a reaction to an organ transplanted into the patient. The particular target and ligand may be specific to, but not limited to, the type of organ transplant. The ligand may have an affinity for a biological molecule associated with a reaction to an organ transplant. The ligand may have an affinity for a cell marker or markers associated with a reaction to an organ transplant. The marker or markers may be selected such that they represent a viable target on T cells or B cells of the patient's immune system.

In another embodiment, the ligand targets a predetermined target associated with a toxin in the patient. A toxin is defined as any poison produced by an organism including, but not limited to, bacterial toxins, plant toxins, insect toxin, animal toxins, and man-made toxins. The particular target and ligand may be specific to, but not limited to, the type of toxin. The ligand may have an affinity for the toxin or a biological molecule associated with a reaction to the toxin. The ligand may have an affinity for a cell marker or markers associated with a reaction to the toxin. A bacterial toxin target may be, for example, one of Cholera toxin, Diphtheria toxin, and Clostridium botulinus toxin. An insect toxin may be, for example, bee venom. An animal toxin may be, for example, snake toxin, for example, Crotalus durissus terrificus venom.

In another embodiment, the ligand targets a predetermined target associated with a hormone-related disease. The particular target and ligand may be specific to, but not limited to, a particular hormone disease. The ligand may have an affinity for a hormone or a biological molecule associated with the hormone pathway. The ligand may have an affinity for a cell marker or markers associated with the hormone disease. For estrogen-related disease or conditions, a predetermined target may be, for example, estrogen or cell surface marker or markers such as, for example, estrogen receptor. For human growth hormone disease, the predetermined target may be, for example, human growth hormone.

In another embodiment, the ligand targets a predetermined target associated with non-cancerous disease material. The particular target and ligand may be specific to, but not limited to, a particular non-cancerous disease material. The ligand may have an affinity for a biological molecule associated with the non-cancerous disease material. The ligand may have an affinity for a cell marker or markers associated with the non-cancerous disease material. For Alzheimer's disease, a predetermined target may be, for example, amyloid B protein and its deposits, or apolipoprotein and its deposits.

In another embodiment, the ligand targets a proteinaceous pathogen. As an example, for prion diseases also known as transmissible spongiform encephalopathies, a predetermined target may be, for example, Prion protein 3F4.

In an embodiment, the nanoparticle is targeted to a cancer cell. In another embodiment, the particles will localize to a tumor, such as a metastatic tumor or micrometastases. Types of cancers include, but are not limited to, bladder, lung, breast, melanoma, colon, rectal, non-Hodgkin lymphoma, endometrial, pancreatic, kidney, prostate, leukemia, thyroid, and the like.

B. Methods for Imaging a Diseased Tissue

In some embodiments, the presently disclosed subject matter provides a method of imaging a diseased tissue, the method comprising: (a) administering to a tissue or a subject in need of treatment thereof, a therapeutically effective amount of a magnetic nanoparticle having a silica or a gold-silica nanoshell, wherein the magnetic nanoparticle comprises iron oxide crystals prepared from a high-gravity controlled precipitation process; and (b) imaging the magnetic nanoparticle having a silica or a gold-silica nanoshell. In particular embodiments, the imaging is conducted by an imaging technique selected from the group consisting of magnetic resonance imaging, plasmon resonance imaging, x-ray imaging, optical coherence tomography (OCT), and x-ray computed tomography.

### IV. DEFINITIONS

Although specific terms are employed herein, they are used in a generic and descriptive sense only and not for purposes of limitation. Unless otherwise defined, all technical and scientific terms used herein have the same meaning as commonly understood by one of ordinary skill in the art to which this presently described subject matter belongs.

By “disease” or “cell disorder”, it is meant any condition, dysfunction, or disorder that damages or interferes with the normal function of a cell, tissue, or organ.

The term “AMF” (an abbreviation for alternating magnetic field), as used herein, refers to a magnetic field that changes the direction of its field vector periodically, typically in a sinusoidal, triangular, rectangular or similar shape pattern, with a frequency of in the range of from about 80 kHz to about 800 kHz. The AMF may also be added to a static magnetic field, such that only the AMF component of the resulting magnetic field vector changes direction. It will be appreciated that an alternating magnetic field is accompanied by an alternating electric field and is electromagnetic in nature.

The term “coating”, as used herein, refers to a material, combination of materials, or covering of the magnetic nanoparticle, comprising a suitable biocompatible material that serves to affect in vivo transport of the nanoparticle throughout the patient, and facilitates uptake and retention by diseased tissues and cell.

In some embodiments, the term “nanoparticle”, as used herein, refers to a targeted nanoparticle that may comprise a magnetic nanoparticle core, coating, linker, and targeting ligand, that is used to selectively treat tissue by heating in response to an alternating magnetic field (AMF). Additionally, the nanoparticle may comprise a radioactive source or species that may become radioactive when exposed to an appropriate energy source. The nanoparticle may also comprise a chemotherapeutic agent, such as doxorubicin. In some embodiments, a nanoparticle comprises a coating, is attached to a target (such as a cell) by one or more targeting ligands.

The term “cell disorder” or “diseased tissue”, as used herein, refers to tissue or cells associated with cancer of any type, such as bone marrow, lung, vascular, neuro, colon, ovarian, breast and prostate cancer; diseases of the immune system, such as AIDS; pathogen-borne diseases, which can be bacterial, viral, parasitic, or fungal, examples of pathogen-borne diseases include HIV, tuberculosis and malaria; hormone-related diseases, such as obesity; vascular system diseases; central nervous system diseases, such as multiple sclerosis; and undesirable matter, such as adverse angiogenesis, restenosis, amyloidosis, toxins, reaction-by-products associated with organ transplants, and other abnormal cell or tissue growth. The term “ligand”, as used herein, refers to a molecule or compound that attaches to a nanoparticle and targets and attaches to a biological marker.

The terms “linker” or “linker molecule,” as used herein, refer to an agent that targets particular functional groups on a ligand and on a magnetic particle or a coating, and thus forms a covalent link between any two of these.

The term “target”, as used herein, refers to the matter for which deactivation, rupture, disruption or destruction is desired, such as a diseased cell, a pathogen, or other undesirable matter. A marker may be attached to the target.

By “contacting”, it is meant any action that results in at least one molecule of one of the presently disclosed nano- or micro-particles physically contacting at least one cell. It thus may comprise exposing the cell(s) to the particle in an amount sufficient to result in contact of at least one particle with at least one cell. The method can be practiced in vitro or ex vivo, by introducing, and preferably mixing, the compound and cells in a controlled environment, such as a culture dish or tube. The method can be practiced in vivo, in which case contacting means exposing at least one cell in a subject to at least one particle of the presently disclosed subject matter, such as administering the particle to a subject via any suitable route. The method for administration of a magnetic material composition to a subject may include intraperitoneal injection, intravascular injection, intramuscular injection, subcutaneous injection, topical, inhalation, ingestion, rectal insertion, wash, lavage, rinse, or extracorporeal administration into a patient's bodily materials. According to the presently disclosed subject matter, contacting may comprise introducing, exposing, and the like, the particle at a site distant to the cells to be contacted, and allowing the bodily functions of the subject, or natural (e.g., diffusion) or man-induced (e.g., swirling) movements of fluids to result in contact of the particle and cell(s).

The subject treated by the presently disclosed methods in their many embodiments is desirably a human subject, although it is to be understood that the methods described herein are effective with respect to all vertebrate species, which are intended to be included in the term “subject.” Accordingly, a “subject” can include a human subject for medical purposes, such as for the treatment of an existing condition or disease or the prophylactic treatment for preventing the onset of a condition or disease, or an animal subject for medical, veterinary purposes, or developmental purposes. Suitable animal subjects include mammals including, but not limited to, primates, e.g., humans, monkeys, apes, and the like; bovines, e.g., cattle, oxen, and the like; ovines, e.g., sheep and the like; caprines, e.g., goats and the like; porcines, e.g., pigs, hogs, and the like; equines, e.g., horses, donkeys, zebras, and the like; felines, including wild and domestic cats; canines, including dogs; lagomorphs, including rabbits, hares, and the like; and rodents, including mice, rats, and the like. An animal may be a transgenic animal. In some embodiments, the subject is a human including, but not limited to, fetal, neonatal, infant, juvenile, and adult subjects. Further, a “subject” can include a patient afflicted with or suspected of being afflicted with a condition or disease. Thus, the terms “subject” and “patient” are used interchangeably herein.

The “effective amount” of an active agent or drug delivery device refers to the amount necessary to elicit the desired biological response. As will be appreciated by those of ordinary skill in this art, the effective amount of an agent or device may vary depending on such factors as the desired biological endpoint, the agent to be delivered, the composition of the encapsulating matrix, the target tissue, and the like.

Following long-standing patent law convention, the terms “a,” “an,” and “the” refer to “one or more” when used in this application, including the claims. Thus, for example, reference to “a subject” includes a plurality of subjects, unless the context clearly is to the contrary (e.g., a plurality of subjects), and so forth.

Throughout this specification and the claims, the terms “comprise,” “comprises,” and “comprising” are used in a non-exclusive sense, except where the context requires otherwise. Likewise, the term “include” and its grammatical variants are intended to be non-limiting, such that recitation of items in a list is not to the exclusion of other like items that can be substituted or added to the listed items.

For the purposes of this specification and appended claims, unless otherwise indicated, all numbers expressing amounts, sizes, dimensions, proportions, shapes, formulations, parameters, percentages, quantities, characteristics, and other numerical values used in the specification and claims, are to be understood as being modified in all instances by the term “about” even though the term “about” may not expressly appear with the value, amount or range. Accordingly, unless indicated to the contrary, the numerical parameters set forth in the following specification and attached claims are not and need not be exact, but may be approximate and/or larger or smaller as desired, reflecting tolerances, conversion factors, rounding off, measurement error and the like, and other factors known to those of skill in the art depending on the desired properties sought to be obtained by the presently disclosed subject matter. For example, the term “about,” when referring to a value can be meant to encompass variations of, in some embodiments, ±100% in some embodiments±50%, in some embodiments±20%, in some embodiments±10%, in some embodiments±5%, in some embodiments±1%, in some embodiments±0.5%, and in some embodiments±0.1% from the specified amount, as such variations are appropriate to perform the disclosed methods or employ the disclosed compositions.

Further, the term “about” when used in connection with one or more numbers or numerical ranges, should be understood to refer to all such numbers, including all numbers in a range and modifies that range by extending the boundaries above and below the numerical values set forth. The recitation of numerical ranges by endpoints includes all numbers, e.g., whole integers, including fractions thereof, subsumed within that range (for example, the recitation of 1 to 5 includes 1, 2, 3, 4, and 5, as well as fractions thereof, e.g., 1.5, 2.25, 3.75, 4.1, and the like) and any range within that range.

### EXAMPLES

The following Examples have been included to provide guidance to one of ordinary skill in the art for practicing representative embodiments of the presently disclosed subject matter. In light of the present disclosure and the general level of skill in the art, those of skill can appreciate that the following Examples are intended to be exemplary only and that numerous changes, modifications, and alterations can be employed without departing from the scope of the presently disclosed subject matter. The synthetic descriptions and specific examples that follow are only intended for the purposes of illustration, and are not to be construed as limiting in any manner to make compounds of the disclosure by other methods.

### Example 1

### Materials and Methods for Preparation of Iron Oxide Nanoparticles

Anhydrous iron(III) chloride (FeCl₃) and anhydrous citric acid were purchased from GCE laboratory chemicals. Iron(II) chloride tetrahydrate (FeCl₂.4F₂O) and ammonia solution (25%) were purchased from Uni Chem Chemical and Merck Co (Whitehouse Station, N.J.), respectively. Tetraethylorthosilicate (TEOS), tetrakis(hydroxymethyl)phosphonium chloride (THPC), 3-aminopropyltetramethylsilicate (APTMS), sodium hydroxide, hydroxylamine (50% in H₂O), potassium carbonate and chloroauric acid tetrahydrate (HAuCl₄.4H₂O) were obtained from Sigma-Aldrich. Ammonium hydroxide solution (30%) was purchased from Merck Company. All the solvents and reagents were of analytical grade and used without further purification.

Magnetite (Fe₃O₄) particles were prepared in a small scale HGCP platform via co-precipitation method. Iron precursor solution was freshly prepared by 24.4 g of anhydrous FeCl₃ and 14.9 g of FeCl₂.4H₂O in 500 mL of 0.74 M hydrochloric acid and kept under inert gas protection at 90° C. Under continuous flow of nitrogen gas, excess 25% ammonia solution was added with vigorous stirring. The reaction mixture turned black immediately and 40 mL of 0.24 M citric acid solution was added. Reaction was continued for 1 hour and magnetite particles were allowed to settle. The supernatant was decanted and settlement was isolated by centrifugation. The particles were washed several times by solvent/anti-solvent precipitation with water and acetone to achieve dispersion at pH 6-8. The trace of acetone was removed under reduced pressure at 60° C. for 15 minutes before the dispersion were treated hydrothermaily at 130° C. for 5 hours. The final dispersion was placed under ultrasonic to ensure well dispersion. The final products were purged by argon gas and kept in a sealed bottle to prevent oxidation of Fe₃O₄ to Fe₂O₃.

### Example 2

### Synthetic Coating Procedures

### Synthesis of Gold Colloid Suspension

A gold colloid suspension was synthesized by the combination of aqueous sodium hydroxide, chloroauric acid and tetrakis(hydroxymethyl)phosphonium chloride (THPC). Duff, et al., Langmuir 1993, 9 (9), 2301-2309. Aqueous sodium hydroxide (1 M, 600 mL) and aqueous THPC (1.2 mM, 2 mL) were added to 90 mL of deionized water and stirred rapidly for ten minutes. Chloroauric acid (1 wt %, 3.4 mL) was quickly added and the solution immediately turned dark brown. The gold colloid solution was aged at 4° C. for at least two weeks before use. Brinson, et al., Langmuir 2008, 24 (24), 14166-14171.

### Synthesis of MION Iron Oxide-Silica-Gold Nanoshells

Magnetite particles synthesized using the HGCP-HTA process as described hereinabove were coated with a thin layer of silica using a modified Stöber method in order to provide an intermediate layer for the binding of gold in future steps. Stöber, et al., Journal of Colloid and Interface Science 1968, 26 (1), 62.

MION particles and 30% ammonium hydroxide were added consecutively to a solution of ethanol and water (3:1 v/v). The nanoparticle mixture was sonicated for 15 minutes followed by the addition of tetraethylorthosilicate (TEOS). The solution was quickly vortexed and the reaction vial was placed on a mechanical rocker (80 rpm) overnight. The silica-coated particles were washed three times with ethanol by centrifugation at 3000 RCF to remove any excess TEOS and were redispersed in water. The particles were sonicated for five minutes to ensure homogenous distribution in the subsequent amino-termination step.

The silica coating was amino-terminated by the addition of 3-aminopropyltetramethylsilicate (APTMS) and Triton X-100 or, in some embodiments, water. Following the addition of APTMS, the particles were agitated overnight on a mechanical rocker. The amino-terminated nanoparticles were washed three times in ethanol for 30 minutes by centrifugation at 3000 RCF before proceeding to gold seeding of the silica surface and subsequent gold plating.

The silica surface was seeded using the gold THPC colloid suspension described hereinabove. Duff, et al., Langmuir 1993, 9 (9), 2301-2309. The THPC precursor solution was diluted with 1.8 mM aqueous K₂CO₃ and sonicated for two minutes. Aqueous sodium chloride (1 M) and the amino-terminated nanoparticles were quickly added to the solution and sonication was continued for an additional two minutes. Brinson, et al., Langmuir 2008, 24 (24), 14166-14171. The solution was then allowed to sit overnight at 4° C. The gold seeded nanoparticles were washed one time with 1.8 mM aqueous K₂CO₃ by centrifugation at 3000 RCF and three times using a permanent magnet. The particles were subsequently redistributed in K₂CO₃.

A 1% HAuCl₄ solution in aqueous potassium carbonate and, in some embodiments, a few microliters of a 3% TWEEN 20 solution were added to the gold seeded nanoparticles. The solution was vortexed then allowed to sit for 30 minutes before adding the reducing agent. While vortexing, hydroxylamine (50% in H₂O) was added and the mixture immediately turned dark purple. The solution was then rocked overnight at room temperature. The nanoparticles were washed three times with 1.8 mM aqueous potassium carbonate using a permanent magnet. The iron oxide-silica-gold nanoshells (e.g., AuSi-MIONs) were then redistributed in 1.8 mM aqueous K₂CO₃ and stored at 4° C.

### Example 3

### Sample Characterization

### Particle Size Analysis by Dynamic Light Scattering (DLS)

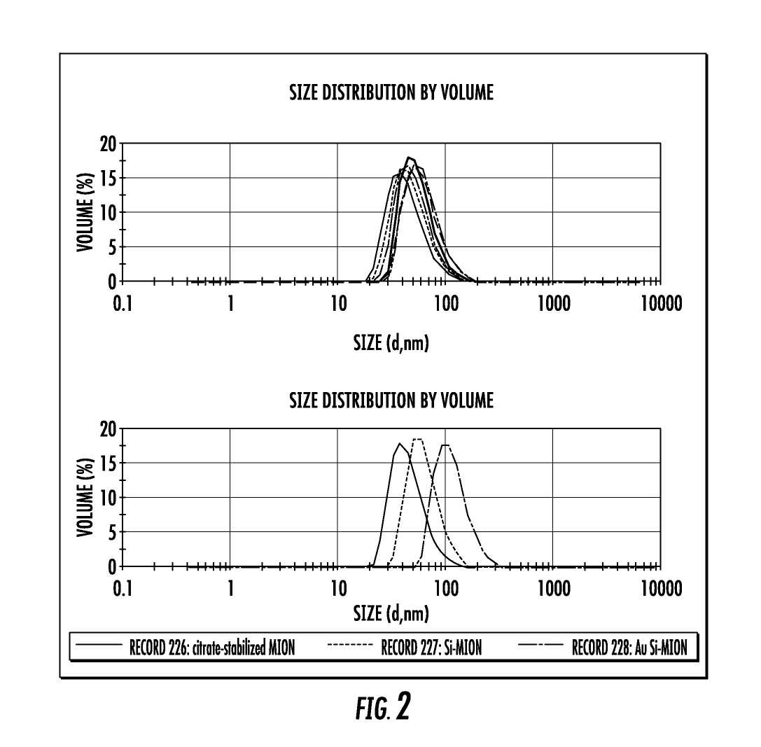

The hydrodynamic diameters of the uncoated and the coated particles were measured on a Zetasizer Nano (Malvern Instruments, Worcestershire, UK) in 1.8 mM K₂CO₃ (see, e.g., FIG. 2 for coated particles).

### Transmission Electron Microscopy (TEM)

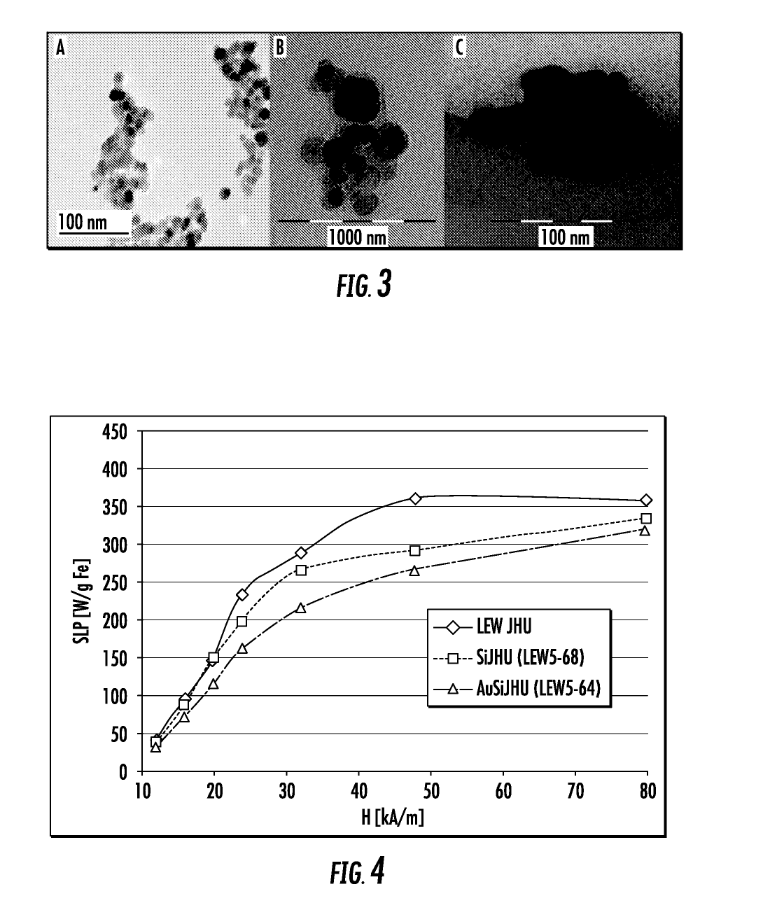

The TEM images were acquired on a Philips EM 420 transmission electron microscope equipped with a SIS Megaview III CCD digital camera (see, e.g., FIG. 3). The specimens were prepared by placing 10 μL of suspension containing the appropriate sample in 100 μL of water onto a carbon coated copper grid (Ted Pella). The grids were allowed to dry at room temperature for 24 hours before use.

### Small Angle Neutron Scattering

Small angle neutron scattering (SANS) data were acquired on the CHRNS 30 m SANS (NG3) instrument at the National Institute of Standards at Technology Center for Neutron Research (NCNR) in Gaithersburg, Md. Data were taken with 0.84-nm wavelength neutrons in transmission and spanned the range of scattering vectors (Q) from 3×10⁻⁵ to 5×10⁻¹ Å⁻¹ using three detector settings. Samples were run in water, which has a scattering length density (SLD) of −5×10^{-7 1}/Å², in cells with 1 mm quartz windows. Specific loss power measurements were performed using a modified solenoid induction coil that produces a homogenous magnetic field encompassing the entire sample volume. A fiber optic temperature probe (FISO Technologies, Quebec City, Canada) was inserted into a 12-mm polystyrene tube containing 1 mL of nanoparticle suspension. The rise in temperature was recorded for each sample over a range of amplitudes from 4-94 kA/m at a fixed frequency of 150 kHz±5 kHz. The SLP was estimated from the slope, ΔT/Δt, of the time-temperature curve using methods described previously. Bordelon, et al., Journal of Applied Physics 109, 12904.1-12904.8 (2011).

### Heating Measurements in Alternating Magnetic Fields

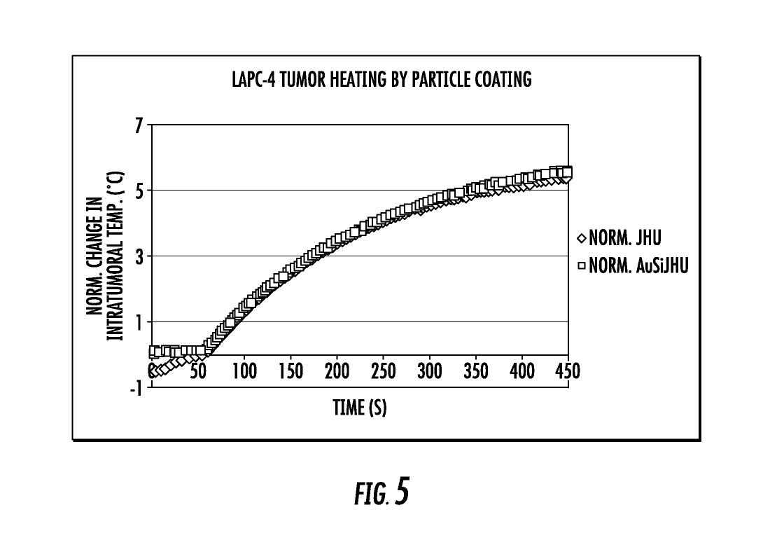

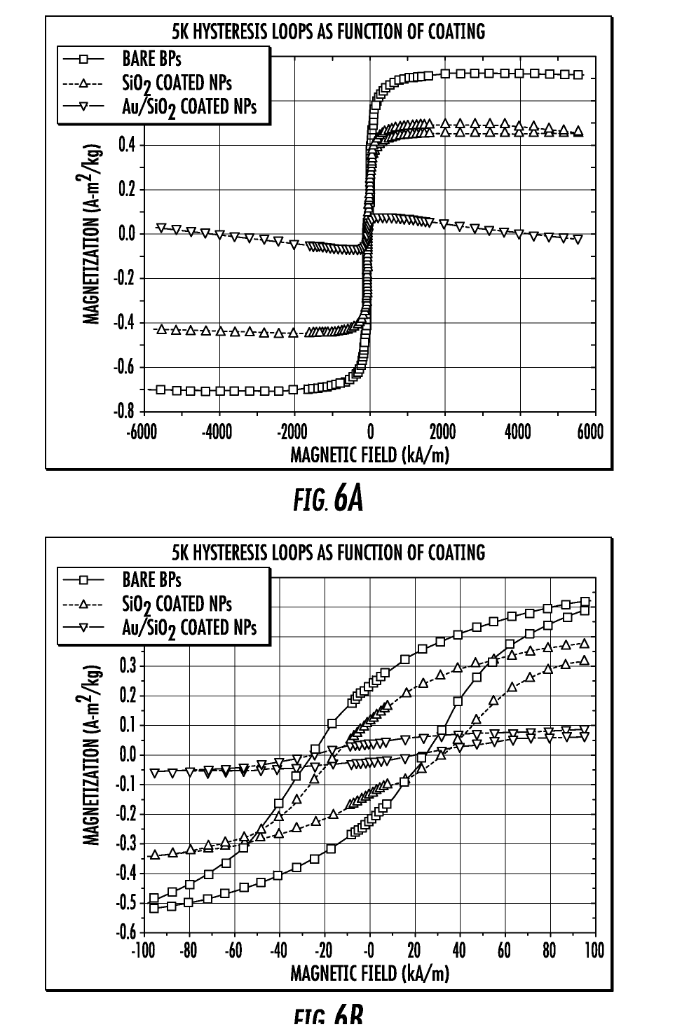

The heating measurements were performed by the Department of Radiation Oncology & Molecular Radiation Sciences in JHU (see, e. g., FIGS. 3 and 4). Equipment used for specific loss power (SLP) and in vivo heating experiments was previously described. Kumar, A., et al., International Journal of Hyperthermia 2013, 29 (2), 106-120.

### Superconducting Quantum Interference Device (SQUID) Magnetometry

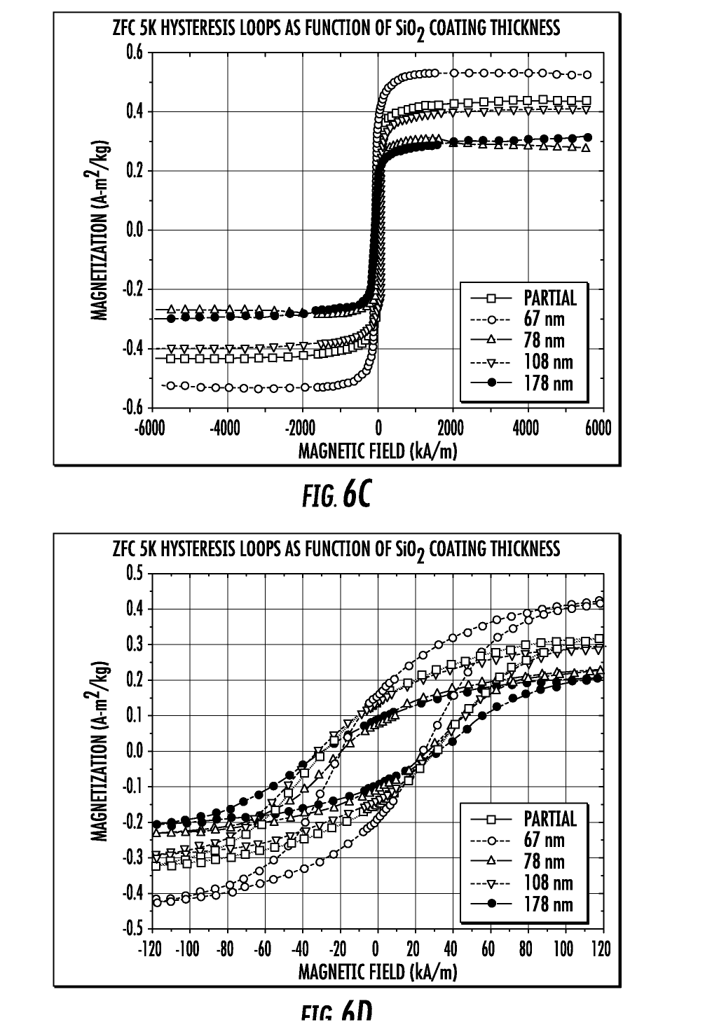

SQUID magnetometry measurements (see, e.g., FIG. 6) were performed at the National Institute of Standards and Technology (NIST). Hysteresis loops were obtained using a MPMS SQUID magnetometer (Quantum Design) in a Kel-F liquid capsule holder (LakeShore Cryotronics) over the field range of ±3.98 MA/m.

### Computed Tomography

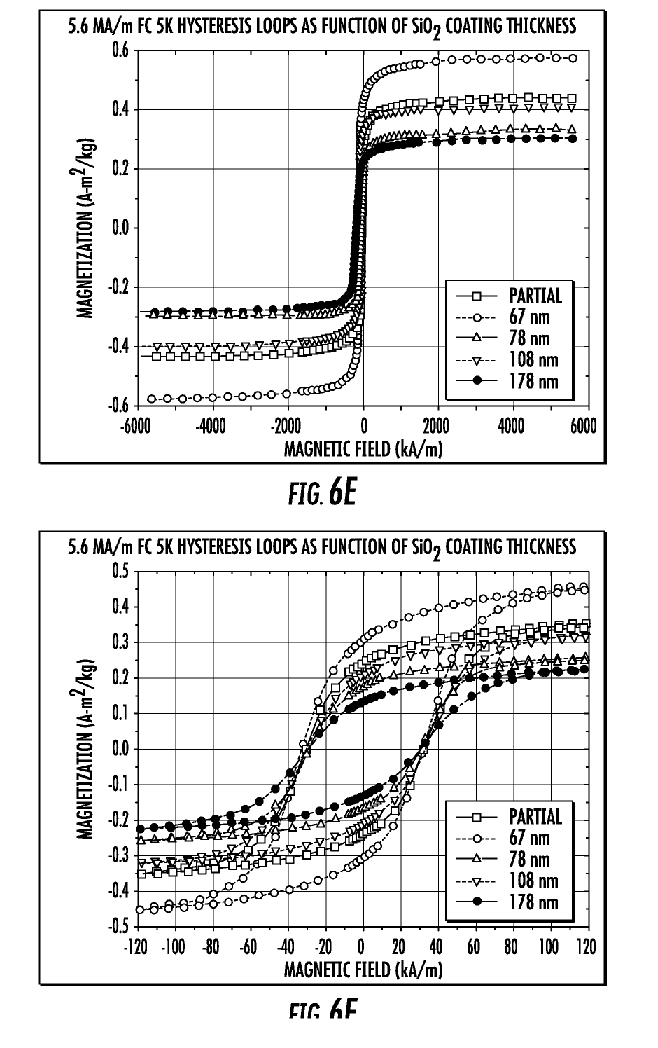

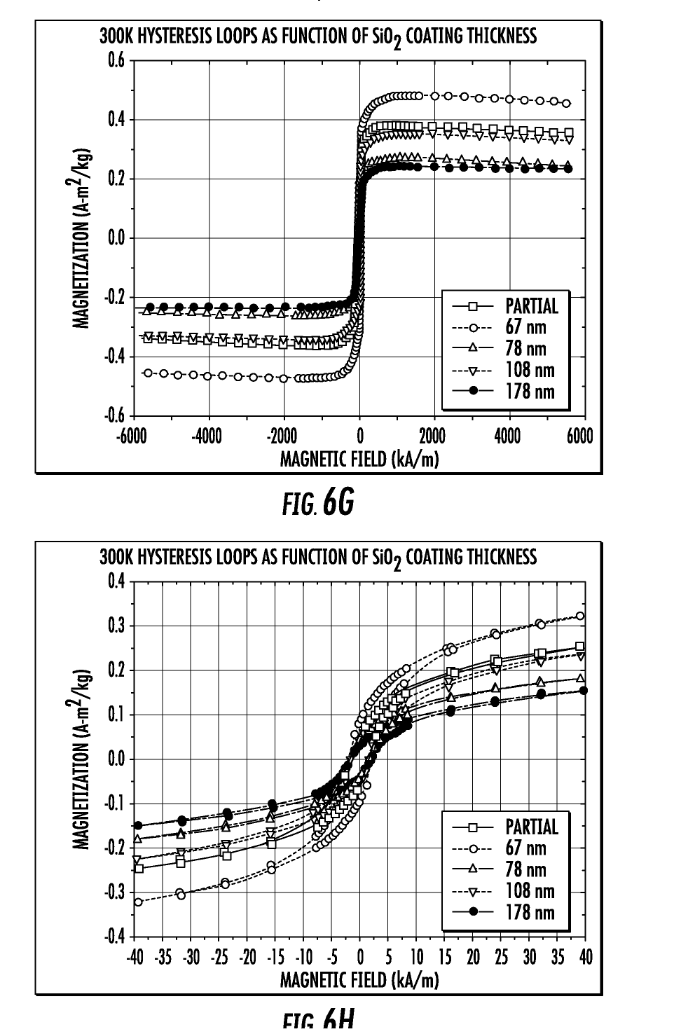

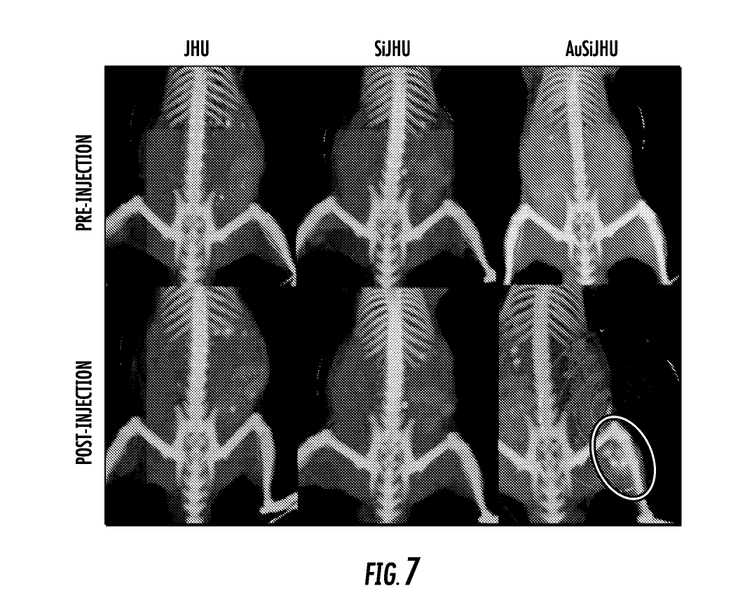

Using the Small Animal Radiation Research Platform (SARRP) in the Department of Radiation Oncology & Molecular Radiation Sciences at JHU, computed tomography (CT) images were obtained using MION, SiMION and AuSiMION nanoparticles. Nanoparticles were injected intratumorally in mice bearing LAPC4 tumors in their right hind legs. Images shown in FIG. 7 demonstrate the ability of AuSiMIONs to act as a CT contrast enhancer.

### In Vivo CT Imaging of LAPC-4 Tumours

Mice bearing prostate tumour xenografts were anesthetized using an isofluorane chamber. The mouse was then moved to the SARRP stage and was maintained under anesthesia using a nose cone. Following an initial CT image obtained at 65 kV and 0.7 mA, the mouse was injected intratumourally with a solution of MIONs (5.5 mg iron per cm³ of tumour). A second CT image was immediately acquired and the data was reconstructed using ImageJ software.

### AMF Hyperthermia Therapy

Mice bearing LAPC-4 prostate cancer xenografts on the right hind flank were subjected to AMF hyperthermia therapy following X-ray CT imaging of intratumourally injected MIONs and AuSi-MIONs. The system described previously, Kumar, et al., International Journal of Hyperthermia 29, 106-120, (2013), consists of an 80 kW power supply (PPECO, Watsonville, Calif.), an external capacitance network (AMF Life Systems, Inc., Auburn Hills, Mich.) adjusted for stable oscillation at 150±5 kHz and a solenoid coil. A custom built water jacket inserted into the AMF coil is used to maintain the physiological body temperature of the mice during therapy. Intratumoural, rectal and contralateral skin temperatures were monitored with fiber optic temperature probes (FISO, Inc., Quebec, Canada). Temperatures were recorded at one-second intervals. The anesthetized mouse was placed in water jacket and inserted into the AMF coil. Therapy was conducted at 150 kHz±5 kHz and 40 kA/m for 20 minutes. Dennis, et al., Nanotechnology 20, 395103 (2009). The temperature of the water jacket was varied in order to maintain mouse body temperature in the range of 40-42° C. during therapy.

### Heating Via Laser

Heating rates of MIONs and AuSi-MIONs via laser excitation were compared in solution using a 5.5 W laparoscopic laser directed at the nanoparticle solutions. The increases in temperature were monitored using a FLIR thermal imaging camera and SARs were normalized based on iron content.

### Example 4

### Further Methods and Characterization

### Synthesis of Gold Colloid Suspension

Aqueous sodium hydroxide (1 M, 600 mL) and aqueous THPC (1.2 mM, 2 mL) were added to 90 mL of deionized water and stirred rapidly for ten minutes. Jackson, et al., European Journal of Radiology 75, 104-109, (2010). Chloroauric acid (1 wt %, 3.4 mL) was quickly added and the solution immediately turned dark brown. The solution was stored at 4° C.

### Gel Phantoms

Phantoms were made for CT and MR imaging using agar solutions. A solution (1% agar in deionized water) was heated to boiling and mixed with the appropriate amount of stock MION suspension in 200 μL, eppendorf tubes to a final volume of 200 μL. K₂CO₃ (1.8 mM) was added to AuSi-MION phantoms. Samples were cooled at room temperature until the gel solidified, and were stored at 4° C.

### T₂-Weighted MR Imaging

T₂-weighted images of gel phantoms containing MIONs ranging from 0 μg Fe/mL to 80 μg Fe/mL were obtained using a Bruker 9.4T horizontal bore spectrometer using spin-echo sequence parameters: repetition time (TR)=4000 ms, echo time (TE)=4, 8, 12, 16, 20, 24, 28 and 32 ms, slice thickness=40 mm, resolution=128×128 pixels. Images were reconstructed and analyzed with ImageJ software (available from the National Institutes of Health).

### X-Ray CT Imaging

X-ray computed tomography (CT) imaging was performed on gel samples loaded with MION concentrations ranging from 0 mg Fe/mL to 7 mg Fe/mL. CT imaging was performed at 65 kV and 0.7 mA with a SARRP (xStrahl Ltd., Surrey, UK) system. Images were reconstructed using 1800 projections and Hounsfield units were calculated for each MION concentration with ImageJ software.

### Tumour Model

5-7 week old male nude mice (Hsd: Athymic Nude-Foxn1^{nm}, Harlan Labs, Indianapolis, Ind.) weighing approximately 20 grams were used for animal experiments. All experiments were conducted according to protocols approved by the Johns Hopkins Institutional Animal Care and Use Committee. Xenograft tumours were obtained by injecting 5×10⁶ LAPC-4 cells subcutaneously in the thigh of mice. Tumour volume was estimated from caliper measurements in three orthogonal directions. Mice were used for experiments once tumour volumes measured 0.15±0.03 cm³.

### Confocal Imaging

Following CT imaging and/or AMF hyperthermia therapy, mice were sacrificed and tumours were excised. Tumours were fixed for at least 48 hours in 10% formalin solution before being embedded in paraffin. The paraffin blocks were sectioned and stained with hematoxylin and eosin (H&E), Prussian blue, or silver enhancer. The histological sections were then examined under a Nikon Eclipse 80i microscope (Nikon Instruments, Inc., Melville, N.Y.). Whole-slice images were reconstructed from multiple images obtained at 4× magnification. Magnified images were obtained with a 20× objective.

### Example 5

### Synthesis and Characterization of Multifunctional Core-Shell Magnetic Nanoparticles for Cancer Theranostics

Multifunctional nanoparticle platforms that enable both imaging and therapeutic applications have become extremely popular in recent years. The use of gold in these agents is prevalent given its many advantages for functionalization, imaging and therapy. Bardhan, et al., Adv. Funct. Mater. 19, 3901-3909, (2009); McCarthy, et al., Small 6, 2041-2049 (2010); and Santra, et al., Small 5, 1862-1868 (2009).

To date, no group has synthesized gold/silica/iron oxide nanoparticles that are useful for imaging while retaining heating efficiency for alternating magnetic field (AMF) hyperthermia therapy. The presently disclosed subject matter provides, in some embodiments, the synthesis, characterization and theranostic evaluation of a new core-shell magnetic nanoparticle platform, which shows great potential for combined computed tomography (CT), magnetic resonance imaging (MRI), AMF hyperthermia therapy and photothermal ablation therapy. This gold/silica-coated magnetic iron oxide nanoparticle (AuSi-MION) construct demonstrated utility as a dual CT/MRI contrast agent over a wide range of iron concentrations, retained the magnetic properties of the iron oxide cores making in vivo AMF hyperthermia therapy achievable, and demonstrated a rapid increase in temperature upon laser irradiation. Further development of this construct presents an opportunity for simultaneous cancer therapy, tumour monitoring and nanoparticle tracking.

Magnetic nanoparticles are extremely versatile, having been used for biomedical applications, Pankhurst, et al., Journal of Physics D: Applied Physics 36, R167 (2003), such as cell tracking, Lewin, et al., Nat Biotech 18, 410-414 (2000), drug and gene delivery, McBain, et al., International Journal of Nanomedicine 3 (2008), MRI contrast and AMF hyperthermia therapy, Dennis, et al., Nanotechnology 20, 395103 (2009); Ivkov, et al., Clinical Cancer Research 11, 7093S-7103S, (2005); however, there has been a recent push to develop multifunctional MIONs for the simultaneous detection and treatment of cancer. Bardhan, et al., Adv. Funct. Mater. 19, 3901-3909 (2009); DeNardo, et al., Journal of Nuclear Medicine 48, 437-444 (2007); Gobin, et al., Nano Letters 7, 1929-1934 (2007); O'Neal, et al., Cancer Letters 209, 171-176 (2004); and Lim and Majetich, Nano Today 8, 98-113 (2013).

The development of a theranostic nanoparticle presents an exciting opportunity to advance disease management, allowing for initial diagnosis via imaging, subsequent therapy and ultimately treatment monitoring using a single construct. The use of MIONs for theranostic constructs is ideal given that they are responsive to magnetic fields and thus are inherently MRI contrast agents. When synthesized with appropriate magnetic anisotropy energy, MIONs in AMFs can produce localized, intense heat, which broadly affects multiple cell processes and proteins in ways that complement the DNA-damaging effects of radiation and chemotherapies. Hildebrandt, et al., Critical Reviews in Oncology Hematology 43, 33-56 (2002).

Gold is especially attractive as a coating material for MIONs because its biocompatibility has been demonstrated in human clinical trials and it provides a convenient surface for chemical conjugation of anti-cancer agents or targeting moieties through thiol group linkage. Shukla, et al., LANGMUIR 21, 10644-10654 (2005). Due to its high molecular weight, gold provides x-ray contrast to enhance CT imaging, and it is a known radiation enhancing material. Herold, et al., International Journal of Radiation Biology 76, 1357-1364 (2000). Gold also displays optically responsive properties by plasmon resonance to provide optical imaging or heating depending upon the wavelength of the incident light. Willets and Duyne, Annual review of Physical Chemistry 58, 267-297 (2007). Thus, the addition of a gold coating greatly enhances the imaging and therapeutic potential of MIONs.

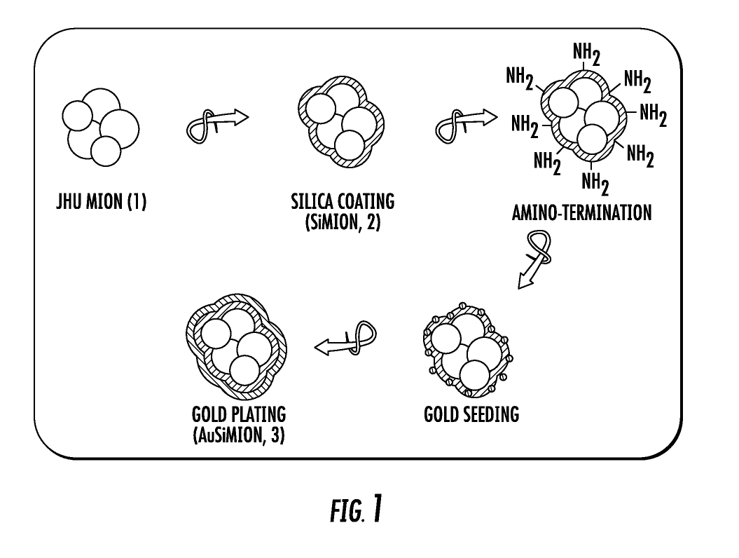

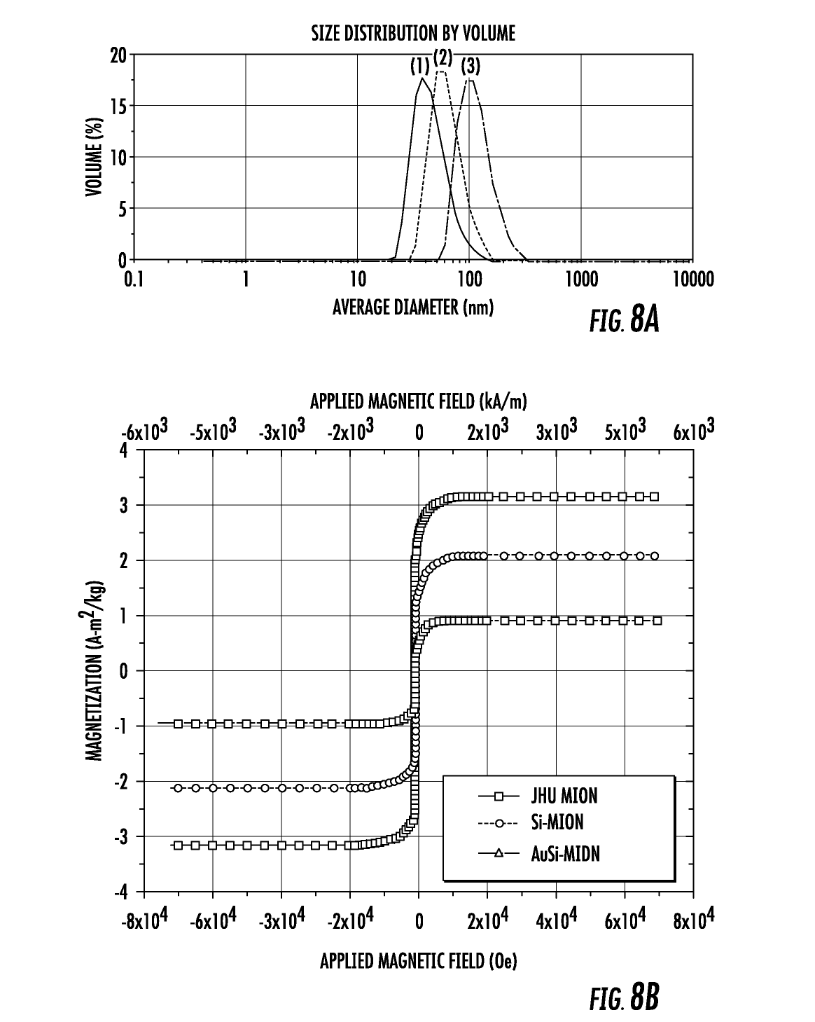

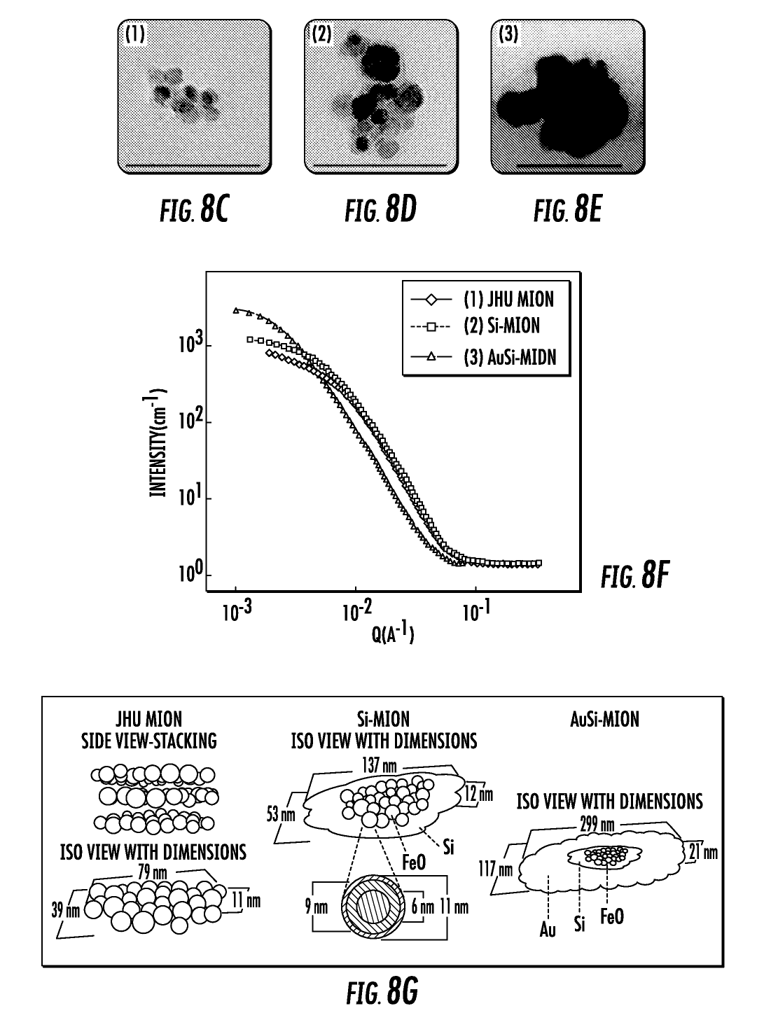

The presently disclosed subject matter provides the synthesis and characterization of a novel gold- and silica-coated theranostic MION construct (FIG. 1, AuSi-MION, 3) along with initial in vivo proof-of-concept studies. Citrate-stabilized magnetic iron oxide cores (MIONs, 55 nm, 1) were coated with silica using a modified Stöber method, Stöber, et al., Journal of Colloid and Interface Science 1968, 26 (1), 62, to form Si-MIONs (2). The reaction was easy to control, as the thickness of the silica layer deposited on the cores increased with increasing equivalents of tetraethylorthosilicate (TEOS). Dynamic light scattering (DLS) confirmed an increase in hydrodynamic diameter of the Si-MIONs to 75 nm (FIG. 8a). The addition of 3-aminopropyltrimethoxysilane (APTMS) to Si-MIONs resulted in the formation of amino-terminated Si-MIONs. Amino-terminated Si-MIONs were seeded with a colloidal gold solution consisting of 1-2 nm gold nanoparticles, which were absorbed by the amine-terminated surface of the Si-MIONs. The reduction of chloroauric acid by hydroxylamine in the presence of the seeded nanoparticles caused the small masses of gold deposited by the seeding process to grow and converge into a solid gold shell forming AuSi-MIONs (3, 140 nm).

Using a superconducting quantum interference device (SQUID) magnetometer, the magnetic hysteresis loops of MIONs, Si-MIONs and AuSi-MIONs were compared and results were normalized based on solid content (FIG. 8b). The results are consistent with the fact that the iron oxide content of the AuSi-MIONs is about one-third of the solid content, resulting in a reduction of the measured magnetic contribution compared to the MIONs. The MIONs were also characterized using transmission electron microscopy (TEM, FIG. 8c). MIONs (1) are multicrystalline cores composed of magnetite crystals that range from 7-10 nm in diameter. Following silica coating, the silica layer encasing the magnetite core was plainly visible and measured about 6-8 nm in thickness. This result is consistent with the size increase to 75 nm upon coating with silica (Si-MIONs, 2, FIG. 8c). It is also clear that the silica is not only coating the outside of the MION, but it is also intercalating into the core of the nanoparticle, coating individual magnetite crystals. The AuSi-MION TEM (3, FIG. 8c) showed an electron dense coating covering the entire surface of the particle consistent with a gold shell.

Small angle neutron scattering (SANS) was employed to further study the interesting physical characteristics of these nanoparticles (FIG. 8d). Glinka, et al., Journal of Applied Crystallography 31, 430-445 (1998). The Si-MION and AuSi-MION batches analyzed by SANS were different from those analyzed by TEM. The thickness of the silica layer was purposely increased to reach an average hydrodynamic diameter of 100 nm as confirmed by DLS. These 100 nm Si-MIONs were used to synthesize AuSi-MIONs with an average hydrodynamic diameter of 180 nm.

Modeling was performed by combining multiple models to fit to each data set. Based on TEM images, nanoparticle size varied along the x-, y- and z-axes. Thus, it was hypothesized that a “triaxialellipsoid” model could be fitted to the data. The SANS data for the MIONs (FIG. 8d) were successfully modeled using two separate computational algorithms in SasView and Igor by summing triaxialellipsoid and stacked discs models. Kline, S. Journal of Applied Crystallography 39, 895-900 (2006).

Dimensions and 3D-representations of the fitting analyses can be seen in FIG. 8e. The three axes of the MION were found to be 11, 39 and 79 nm with a scattering length density (SLD) of 8.2×10⁻⁶ Å². While at first there appears to be a discrepancy between DLS and SANS measurements of mean diameter, it is likely that DLS provides an average of two larger particle dimensions. DLS is useful for sizing nanoparticles, however, it assumes that the particles are spherical and tends to provide an averaged dimension for non-spherical particles.

SANS data obtained from the Si-MION and AuSi-MION samples (FIG. 8d) were fit to a core-shell sphere model combined with the triaxialellipsoid model. Analysis of the Si-MION core-shell gave dimensions of 6.2 nm for the core diameter of each individual magnetite crystal (SLD=7.4×10⁻⁶ Å⁻²) and an average of 2 nm of silica (SLD=4.3×10⁻⁶ Å⁻²) surrounding each crystal. The overall dimensions of the particles with the outer silica shell were determined by the triaxialellipsoid model and were found to be 12, 53 and 137 nm with an SLD of 4.3×10⁻⁶ Å⁻². As with the MIONs, DLS measurements (100 nm), which assume spherical particle shape, are likely the average of the two largest dimensions of the triaxialellipsoid (avg=95 nm). AuSi-MION data analysis gave triaxialellipsoid dimensions of 21, 117 and 299 nm with the SLD of the ellipsoid decreasing slightly to 4.1×10⁻⁶ Å². This decrease in SLD with the addition of the gold is not surprising given that the theoretical SLD of gold is slightly lower than that of silica. The SLDs of silica and gold are 4.5×10⁻⁶ Å⁻² and 4.2×10⁻⁶ Å⁻², respectively, causing the neutrons to pass through these layers without a significant difference in scattering angle.

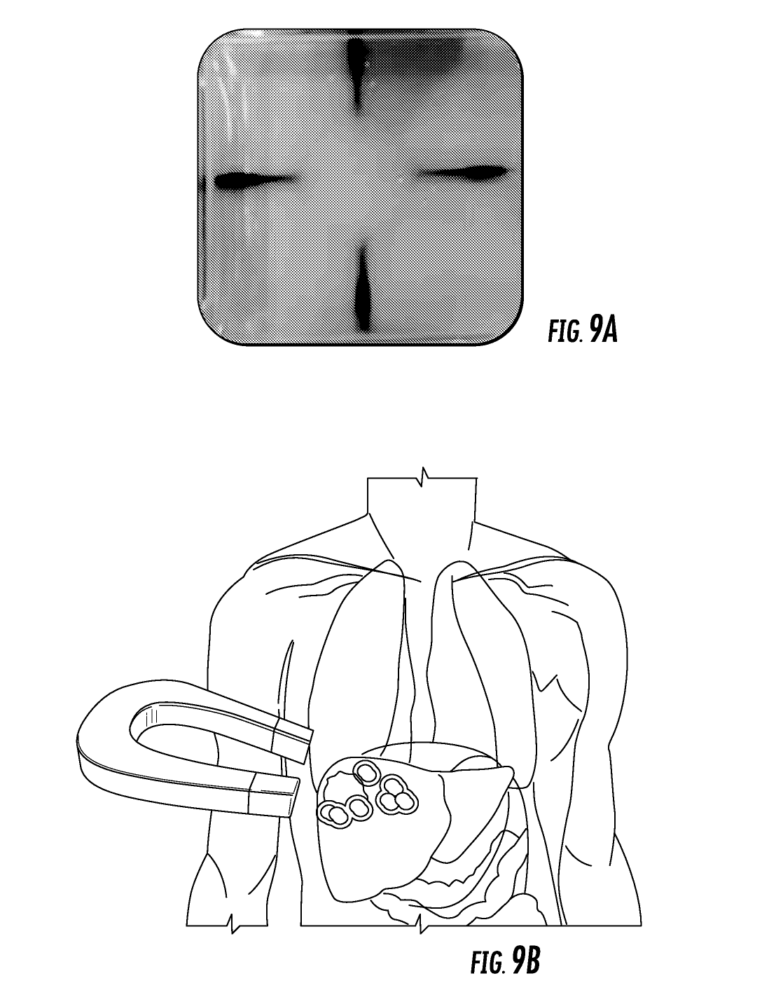

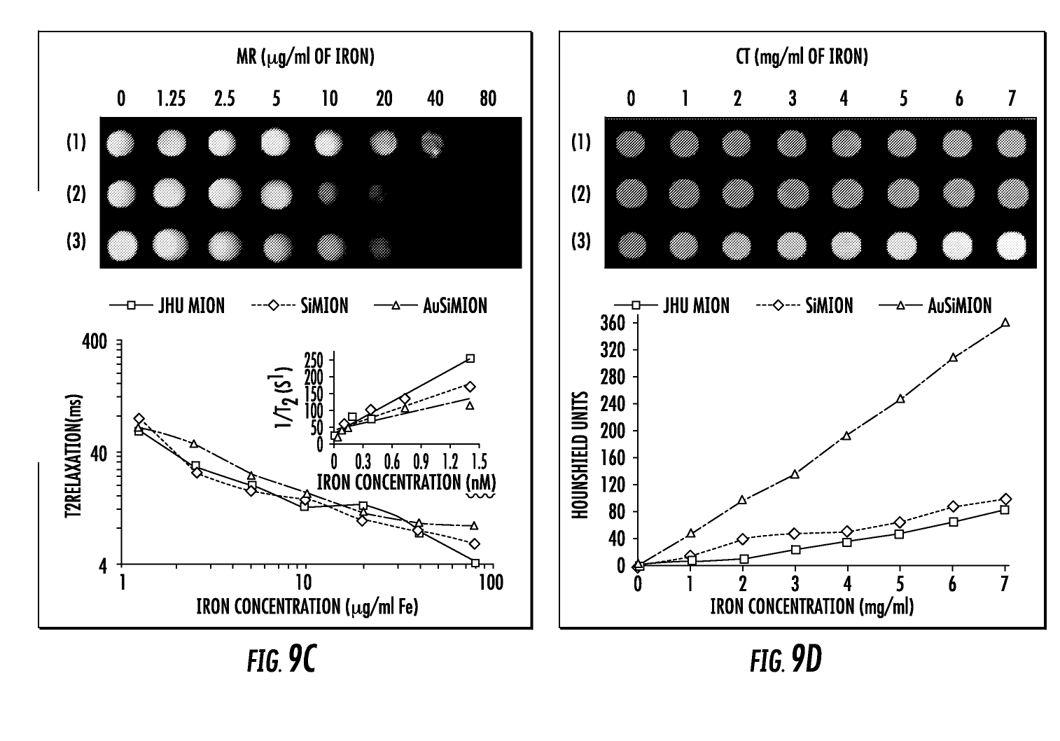

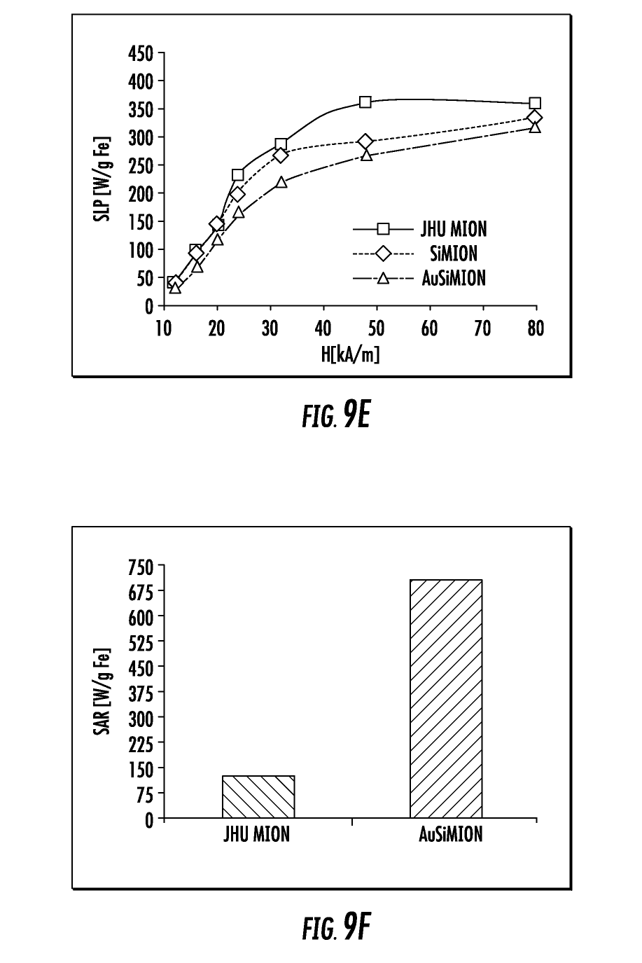

The nanoparticle platform was then evaluated for theranostic potential (FIG. 9). FIG. 9a shows AuSi-MIONs (purple) drawn by a permanent magnet, demonstrating the potential for its magnetic vectorization. This concept is illustrated in FIG. 9b, showing the nanoparticles being guided to a tumour site by an external magnet. To assess the MR contrast capabilities of MIONs 1, 2 and 3, phantoms ranging in iron concentration from 0-80 μg/mL (0-1.4 mM) were imaged using T₂-weighted MRI (FIG. 9c). All three MION types decreased in signal intensity as the iron concentration increased, causing darkening. The darkening effect is due to interaction of protons in water with the magnetic moments of the nanoparticles and indicates that these MIONs are useful for T₂-weighted MR imaging. Further analysis yielded the T₂ relaxation rates (FIG. 9c). The graph inset shows iron concentration (mM) plotted versus the inverse of T₂. The trendline slopes for each nanoparticle give R₂, the transverse relaxivity coefficient, which is a measure of nanoparticle contrast efficiency. The R₂ values were 155, 99 and 68 mM⁻¹s⁻¹ for MIONs 1, 2 and 3, respectively. Feridex, a commercially available iron oxide MRI contrast agent, has an R₂ relaxivity of 98 m M⁻¹s⁻¹. The comparable relaxivities of these MIONs to commercially available contrast agents demonstrates the possibility of using these new constructs for MR imaging.

While magnetite nanoparticles are inherent MRI contrast agents, iron oxide does not create significant contrast in CT scans. Gold (Z=79) has shown superiority over typical iodinated CT contrast agents such as Ultravist®, Bayer Healthcare, LLC, Whippany N.J.). Jackson, et al., European Journal of Radiology 75, 104-109 (2010). MIONs were diluted to a concentration range of 0-7 mg/mL (0-125 mM) based on iron content and compared for their ability to demonstrate CT contrast (FIG. 9d). Signal intensity from the MION (1) and Si-MION (2) phantoms, quantified using the Hounsfield scale, did not increase significantly as iron concentration increased. However, the signal intensity of the AuSi-MIONs increased significantly as the particle concentration increased. At a concentration of 7 mg/mL AuSi-MIONs, the signal intensity reached 361 Hounsfield units. Typical CT contrast agents fall in the range from 100-300 on the Hounsfield scale, making AuSi-MIONs extremely advantageous for inducing CT contrast.

High nanoparticle heating efficiency is necessary at low amplitude for clinical applications in order to avoid non-specific heating. Specific loss power (SLP) is a measurement of amplitude-dependent nanoparticle heating efficiency. As previously reported, Bordelon, et al., Journal of Applied Physics 109, 12904.1-12904.8 (2011). MION SLPs were measured at a fixed frequency of 150±5 kHz and varying amplitudes from 10-80 kA/m, and results were normalized based on iron content (FIG. 9e). AuSi-MION SLP reached 200 W/g Fe at 30 kA/m. The small decreases in magnetic field hysteresis and SLP of the AuSi-MIONs may be due to diamagnetic shielding of the gold shell. Neither the hysteresis loops nor the SLP measurements vary significantly between samples, indicating that neither the synthetic procedures nor the coatings alter the magnetic properties of the core nanoparticles significantly.

The surface plasmon resonance of gold-shelled nanoparticles can result in non-radiative heating when excited by a laser, Huang, et al., Lasers in Medical Science 23, 217-228 2008), therefore, their tunable optical properties and easily functionalized surface make them ideal for cancer therapy via photothermal ablation. Laser heating of MIONs and AuSi-MIONs was compared in solution using a 5.5W laparoscopic laser (FIG. 9f). The laser was directed at solutions of AuSi-MIONs (0.5 mg/mL based on iron content) and MIONs (20 mg/mL iron content) and the increase in temperature was monitored. The specific absorption rate (SAR) of these solutions were normalized by iron content and calculated to be 709 Wg⁻¹Fe for AuSi-MIONs versus 127 Wg⁻¹ Fe for MIONs, confirming that the temperature increase is mostly due to the gold coating and not the iron oxide core. The ability of the AuSi-MIONs to heat rapidly upon laser irradiation validates further investigation of this therapeutic method.

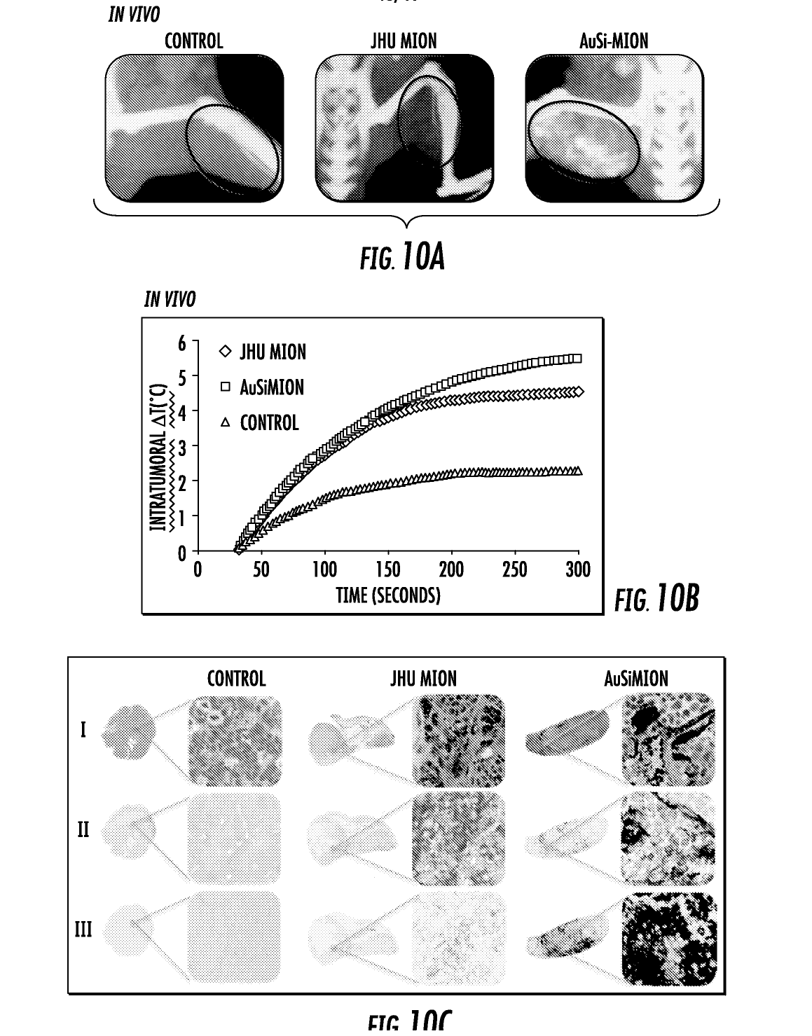

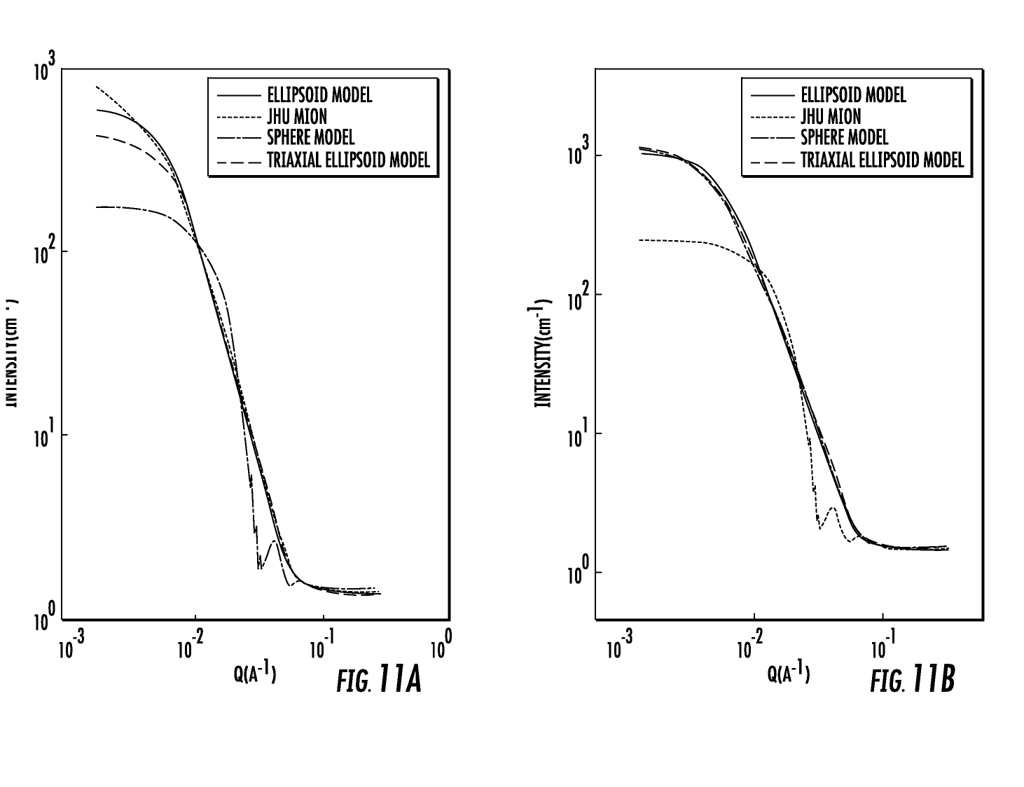

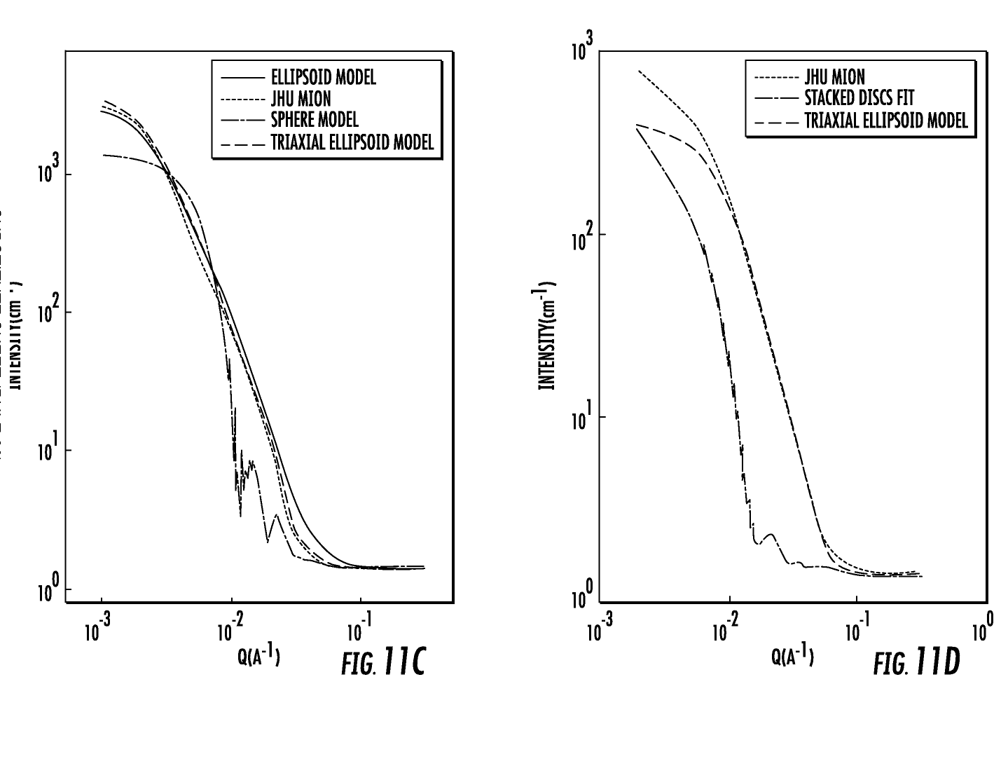

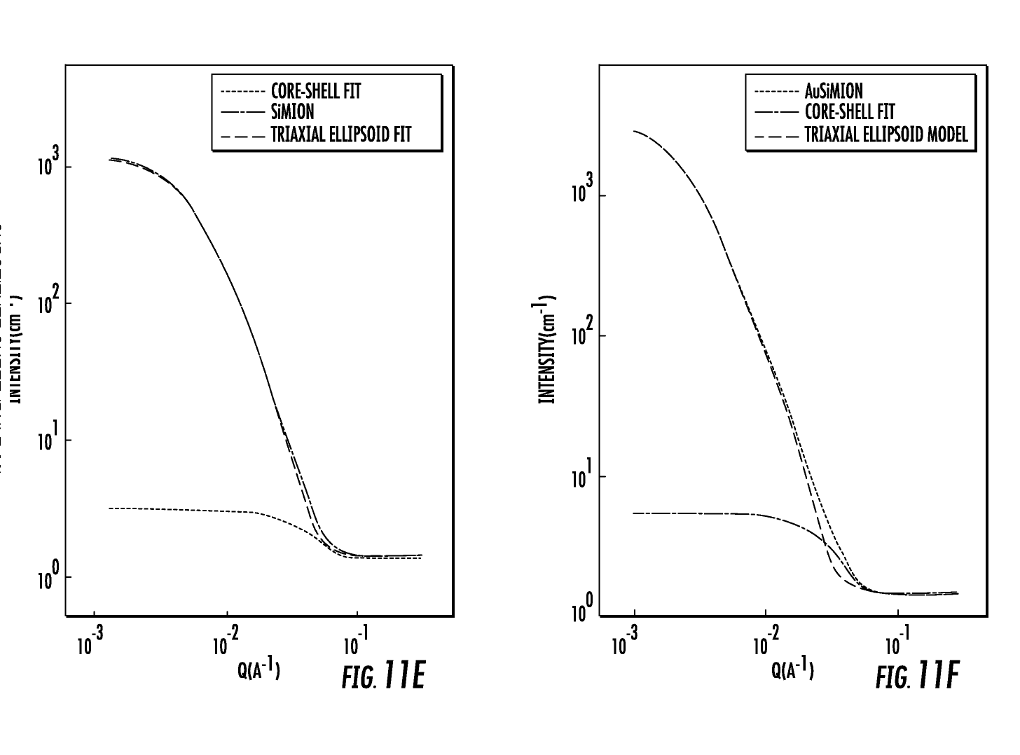

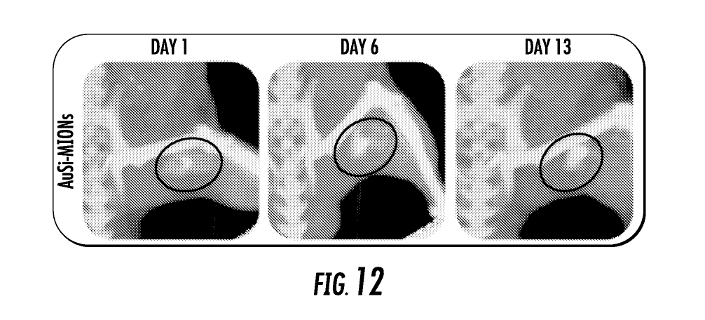

Following initial assessment of the AuSi-MION platform as a dual MRI/CT contrast agent, it was tested in mice bearing LAPC-4 prostate cancer xenograft tumours on their hind legs (FIG. 10a). A control mouse was injected intratumourally with saline only prior to CT imaging (FIG. 10a) and no contrast was observed. Two mice were then injected intratumourally with MIONs or AuSi-MIONs at an iron concentration known to be useful for AMF hyperthermia therapy (5.5 mg Fe/cm³ tumour). Dennis, et al., Nanotechnology 20, 395103 (2009).

CT confirmed the position of the gold-shelled nanoparticles while the MIONs were invisible. A fourth mouse bearing an LAPC-4 xenograft tumour was injected with AuSi-MIONs and monitored by CT for 13 days (FIG. 12). The signal intensity did not decrease, demonstrating that the AuSi-MIONs were retained in the tumour. By utilizing the dual imaging capabilities of AuSi-MIONs, it would be possible to track the nanoparticles for an extended period of time. Initially the particles will have higher local concentrations, which will make MRI unfeasible due to its extreme sensitivity to the iron oxide cores. During this time, CT could track the nanoparticles and monitor tumour volume. However, as the particles disperse over time and concentration decreases, the high sensitivity of MRI to the AuSi-MIONs will enable long-term tracking of the nanoparticles.

Intracellular temperature must be held between 42-46° C. to stimulate tumour cell death by inactivation of normal cellular processes via AMF hyperthermia therapy. DeNardo, et al., Journal of Nuclear Medicine 48, 437-444 (2007). The mice imaged with CT were placed in a water jacket inside a modified solenoid coil capable of producing high amplitude AC magnetic fields. Bordelon, et al., Ieee Transactions on Magnetics 48, 47-52, 2162527 (2012).

Fiber optic probes were used to monitor the intratumoural, body and rectal temperatures of the mice, as well as the temperature of the water jacket. The coil was powered on (150 kHz, 40 kA/m) and heating continued for 20 minutes. The initial tumour heating rates increased rapidly and were nearly identical in mice injected with nanoparticles (FIG. 10b) while only minor heating occurred in the saline control tumour. This demonstrates that the gold plating of the MION cores does not have a detrimental effect on the ability of the particles to heat in AMFs. When introduced to an AMF, AuSi-MIONs can raise intratumoural temperature to the lethal range without overheating the body of the mouse, validating continued development of the AuSi-MION platform.

At 72 hours post-treatment, the mice were euthanized and their tumours were harvested for histological examination (FIG. 10c). Both the MIONs (brown) and the AuSi-MIONs (purple) can be visualized with the H&E stain (row I), while there are no nanoparticles present in the control mouse tissue. Adjacent tumour slices visualised with Prussian blue stain (row II) show that the MIONs and AuSi-MIONs can be co-registered with both the H&E staining, as well as the CT images. Prussian blue stain, which is commonly used to detect the presence of iron in specimens, most likely stained the AuSi-MIONs because the staining procedure uses acids that degrade the gold coating, allowing some iron to leech out of the core. However, only the AuSi-MION tumour sample turned black with the silver enhancement stain (row III), indicating that there is gold present. Silver enhancement stain is frequently used to stain gold nanoparticles and will not stain iron. The gold shell acts as a nucleation site for the silver ions, which are reduced to metallic silver by a reducing agent. Gupta, et al., Analytical chemistry 79, 3810-3820 (2007). This increases the size of the nanoparticle up to 5 orders of magnitude, turning them black and making them easily visible.

A new theranostic nanoparticle construct with dual modality imaging and dual therapeutic potential was synthesized and assessed using phantoms and in an in vivo prostate cancer model. Comprehensive characterization revealed that the magnetic properties of the iron oxide cores were preserved. Because of these highly beneficial magnetic properties, MRI was used to detect as little as 1-2 μg/mL of iron in a sample and AMF hyperthermia therapy with AuSi-MIONs efficiently raised intratumoural temperature by 6° C. in mice while maintaining normal body temperature. The gold shell allowed visualisation of the nanoparticles via CT, heating of tumour cells via photothermal ablation, and will allow facile targeting of this construct to specific receptors in future research.

### REFERENCES

All publications, patent applications, patents, and other references mentioned in the specification are indicative of the level of those skilled in the art to which the presently disclosed subject matter pertains. All publications, patent applications, patents, and other references are herein incorporated by reference to the same extent as if each individual publication, patent application, patent, and other reference was specifically and individually indicated to be incorporated by reference. It will be understood that, although a number of patent applications, patents, and other references are referred to herein, such reference does not constitute an admission that any of these documents forms part of the common general knowledge in the art.
Adair, E. R., and Black, D. R., “Thermoregulatory responses to RF energy absorption,” Bioelectromagnetics 6(Supplement), S17-S38 (2003);
Atkinson, W. J., Brezovich, I. A., Chakraborty, D. P., “Usable frequencies in hyperthermia with thermal seeds,” IEEE Trans. Biomed. Eng. 31, 70-75 (1984);
Black, D. R., [Thermoregulation in the presence of radio frequency fields], Biological and Medical Aspects of Electromagnetic Fields, 3rd Edition, Boca Raton, 215-226 (2006);
Dennis C. L., et al. “Nearly complete regression of tumors via collective behavior of magnetic nanoparticles in hyperthermia,” Nanotechnology 20(39), Article Number 395103 (2009);
Dennis C. L., A. J. Jackson, J. A. Borchers, P. J. Hoopes, R. Strawbridge, A. R. Foreman, J. van Lierop, C. Grüttner, and R. Ivkov, Nanotechnology, 20 (2009) 395103;
Gruettner C, K. Mueller, J. Teller, F. Westphal, A. Foreman, and R. Ivkov, J. “Synthesis and antibody conjugation of magnetic nanoparticles with improved specific power absorption rates for alternating magnetic field cancer therapy,” Journal of Magnetism and Magnetic Materials 311(1), 181-186 (2007);
Grüttner, C., Teller, J., Schütt, W., Westphal, F., Schümichen, C., and Paulke, B. R., Preparation and Characterization of Magnetic Nanospheres for in vivo Application. In Scientific and Clinical Application of Magnetic Carriers (U. O. Hafeli, W. Schütt, J. Teller and M. Zborowski, Eds.), pp. 53-68. Plenum Press, New York, 1997;
Hedayati, M., Thomas, O., Abubaker-Sharif, B., Zhou, H., Cornejo, C., Zhang, Y., Wabler, M., Mihalic, J., Gruettner, C., Westphal, F., Geyh, A., Deweese, T. L., Ivkov, R., “The effect of cell cluster size on intracellular nanoparticle-mediated hyperthermia: is it possible to treat microscopic tumors?,” Nanomedicine (Lond) 8(1), 29-41 (2013);
Ivkov, R., DeNardo, S. J., Daum, W., Foreman, A. R., Goldstein, R. C., Nemkov, V. S., DeNardo, G. L., “Application of high amplitude alternating magnetic fields for heat induction of nanoparticles localized in cancer,” Clin. Cancer Res. 11(19 Suppl), 7093s-7103s (2005);
Jordan, A., Wust. P., Scholz, R., Faehling, H., Krause, J. and Felix, R., [Magnetic Fluid Hyperthermia (MFH)], Scientific and Clinical Applications of Magnetic Carriers, New York, 569-595 (1997);
Kim, J., J. E. Lee, S. H. Lee, J. H. Yu, J. H. Lee, T. G. Park, and T. Hyeon, Adv. Mater., 20 (2008) 478;
Krycka, K. L., A. J. Jackson, J. A. Borchers, J. Shih, R. Briber, R. Ivkov, C. Grüttner, and C. L. Dennis, Journal of Applied Physics, 109 (2011) 07B513.
Kumar, A., Attaluri, A., Mallipudi, R, Cornejo, C., Bordelon, D., Armour, M., Morua, K., DeWeese, T. L., Ivkov, R., “Method to reduce non-specific tissue heating of small animals in solenoid coils,” Int. J Hyperthermia, 29, 106-120 (2013); Nemkov V, et al. “Magnetic field generating inductor for cancer hyperthermia research,” Compel 10(5), 1626-1636 (2011);
Poddar, P., M. B. Morales, N. A. Frey, S. A. Morrison, E. E. Carpenter, and H. Srikanth, J. Appl. Phys., 104 (2008) 063901;
Repetto G, et al. “Neutral red uptake assay for the estimation of cell viability/cytotoxicity,” Nature Protocols 3(7), 1125-1131 (2008);
Rosensweig, R. E., “Heating magnetic fluid with alternating magnetic field,” J. Magnetism and Magn. Materials 252, 370-374 (2002);
Rudershausen S, Grüttner C, Frank M, Teller J, Westphal F: Multifunctional Superparamagnetic Nanoparticles for Life Science Applications. European Cell and Materials 3, 81-83 (2002);
Southern, P., D. Ortega, C. Johansson, and Q. Pankhurst, Talk 35 of the 9th International Conference on the Scientific and Clinical Applications of Magnetic Carriers, Minneapolis, Minn.;
Taketomi, S. and R. D. Shull, J. Appl. Phys., 91 (2002) 8546-8548.
Dennis, C. L.; Jackson, A. J.; Borchers, J. A.; Hoopes, P. J.; Strawbridge, R.; Foreman, A. R.; van Lierop, J.; Gruettner, C.; Ivkov, R., Nearly complete regression of tumors via collective behavior of magnetic nanoparticles in hyperthermia. Nanotechnology 2009, 20 (39).
Dennis C L, Ivkov R; The physics of heat generation using magnetic nanoparticles for hyperthermia. Int. J. Hyperthermia 2013, 29 (8), 715-729.
Hedayati, M.; Thomas, O.; Abubaker-Sharif, B.; Zhou, H.; Cornejo, C.; Zhang, Y.; Wabler, M.; Mihalic, J.; Gruettner, C.; Westphal, F.; Geyh, A.; Deweese, T. L.; Ivkov, R., The effect of cell cluster size on intracellular nanoparticle-mediated hyperthermia: is it possible to treat microscopic tumors? Nanomedicine 2013, 8 (1), 29-41.
Gobin, A. M.; Lee, M. H.; Halas, N. J.; James, W. D.; Drezek, R. A.; West, J. L., Near-infrared resonant nanoshells for combined optical imaging and photothermal cancer therapy. Nano Letters 2007, 7 (7), 1929-1934.
Huang, X.; Jain, P. K.; El-Sayed, I. H.; El-Sayed, M. A., Plasmonic photothermal therapy (PPTT) using gold nanoparticles. Lasers in Medical Science 2008, 23 (3), 217-228.
Lal, S.; Clare, S. E.; Halas, N. J., Nanoshell-Enabled Photothermal Cancer Therapy: Impending Clinical Impact. Accounts of Chemical Research 2008, 41 (12), 1842-1851.
O'Neal, D. P.; Hirsch, L. R.; Halas, N. J.; Payne, J. D.; West, J. L., Photo-thermal tumor ablation in mice using near infrared-absorbing nanoparticles. Cancer Letters 2004, 209 (2), 171-176.
Lechtman, E.; Chattopadhyay, N.; Cai, Z.; Mashouf, S.; Reilly, R.; Pignol, J. P., Implications on clinical scenario of gold nanoparticle radiosensitization in regards to photon energy, nanoparticle size, concentration and location. Physics in Medicine and Biology 2011, 56 (15), 4631-4647.
Oldenburg, A. L.; Hansen, M. N.; Zweifel, D. A.; Wei, A.; Boppart, S. A., Plasmon-resonant gold nanorods as low backscattering albedo contrast agents for optical coherence tomography. Optics Express 2006, 14 (15), 6724-6738.
Duff, D. G.; Baiker, A.; Edwards, P. P., A New Hydrosol of Gold Clusters 0.1. Formation and Particle-Size Variation. Langmuir 1993, 9 (9), 2301-2309.
Brinson, B. E.; Lassiter, J. B.; Levin, C. S.; Bardhan, R.; Mirin, N.; Halas, N. J., Nanoshells Made Easy: Improving Au Layer Growth on Nanoparticle Surfaces. Langmuir 2008, 24 (24), 14166-14171.
Stober, W.; Fink, A.; Bohn, E., Controlled Growth of Monodisperse Silica Spheres in Micron Size Range. Journal of Colloid and Interface Science 1968, 26 (1), 62.
Kumar, A.; Attaluri, A.; Mallipudi, R.; Cornejo, C.; Bordelon, D.; Armour, M.; Morua, K.; Deweese, T. L.; Ivkov, R. Method to reduce non-specific tissue heating of small animals in solenoid coils. International Journal of Hyperthermia 2013, 29 (2), 106-120.
Bordelon, D., Goldstein, R., Nemkov, V., Kumar, A., Jackowski, J., DeWeese, T. L., Ivkov, R., Modified solenoid coil that efficiently produces high amplitude AC magnetic fields with enhanced uniformity for biomedical applications, IEEE Trans. on Magnetics 2012, 48, 47-52.
Bordelon, D., Comejo, C., Gruettner, C., Westphal, F., DeWeese, T. L., Ivkov, R., Magnetic nanoparticle heating efficiency reveals magneto-structural differences when characterized with wide ranging and high amplitude alternating magnetic fields, Journal of Applied Physics 2011, 109, 12904.1-12904.8.
Kut, C., Zhang, Y., Hedayati, M., Zhou, H., Comego, C., Bordelon, D. E., Mihatic, J., Wabler, M., Burghardt, E., Grueltner, C., Geyh, A., Brayton, C., DeWeese, T. L., Ivkov, R., Preliminary study of injury from heating systemically delivered, nontargeted dextran-superparamagnetic iron oxide nanoparticles in mice, Nanomedicine 2012, 7, 1697-1711.
Hedayati, M., Attalluri, A., Bordelon, D., Goh, R., Armour, M., Zhou, H., Comego, C., Wabler, M., Zhang, Y., DeWeese, T., Ivkov, R., New iron-oxide particles for magnetic nanoparticle hyperthermia: an in-vitro and in-vivo pilot study, Proceedings of SPIE 2013, 8584, 858404.
Bardhan, R. et al. Nanoshells with Targeted Simultaneous Enhancement of Magnetic and Optical Imaging and Photothermal Therapeutic Response. Adv. Funct. Mater. 19, 3901-3909 (2009).
McCarthy, J. R., Korngold, E., Weissleder, R. & Jaffer, F. A. A Light-Activated Theranostic Nanoagent for Targeted Macrophage Ablation in Inflammatory Atherosclerosis. Small 6, 2041-2049 (2010).
Santra, S., Kaittanis, C., Grimm, J. & Perez, J. M. Drug/Dye-Loaded, Multifunctional Iron Oxide Nanoparticles for Combined Targeted Cancer Therapy and Dual Optical/Magnetic Resonance Imaging. Small 5, 1862-1868 (2009).
Pankhurst, Q. A., Connolly, J., Jones, S. K. & Dobson, J. Applications of magnetic nanoparticles in biomedicine. Journal of Physics D: Applied Physics 36, R167 (2003).
Lewin, M. et al. Tat peptide-derivatized magnetic nanoparticles allow in vivo tracking and recovery of progenitor cells. Nat Biotech 18, 410-414 (2000).
McBain, S. C., Yiu, H. H. & Dobson, J. Magnetic nanoparticles for gene and drug delivery. International Journal of Nanomedicine 3 (2008).
DeNardo, S. J. et al. Thermal dosimetry predictive of efficacy of In-111-ChL6 nanoparticle AMF-induced thermoablative therapy for human breast cancer in mice. Journal of Nuclear Medicine 48, 437-444 (2007).
Lim, J. & Majetich, S. A. Composite magnetic-plasmonic nanoparticles for biomedicine: Manipulation and imaging. Nano Today 8, 98-113 (2013).
Hildebrandt, B. et al. The cellular and molecular basis of hyperthermia. Critical Reviews in Oncology Hematology 43, 33-56 (2002).
Shukla, R. et al. Biocompatibility of Gold Nanoparticles and Their Endocytotic Fate Inside the Cellular Compartment: A Microscopic Overview. LANGMUIR 21, 10644-10654 (2005).
Herold, D. M., Das, I. J., Stobbe, C. C., Iyer, R. V. & Chapman, J. D. Gold microspheres: a selective technique for producing biologically effective dose enhancement. International Journal of Radiation Biology 76, 1357-1364 (2000).
Willets, K. A. & Duyne, R. P. V. Localized Surface Plasmon Resonance Spectroscopy and Sensing. Annual review of Physical Chemistry 58, 267-297 (2007).
Glinka, C. J. et al. The 30 m Small-Angle Neutron Scattering Instruments at the National Institute of Standards and Technology. Journal of Applied Crystallography 31, 430-445 (1998).
Kline, S. Reduction and analysis of SANS and USANS data using IGOR Pro. Journal of Applied Crystallography 39, 895-900 (2006).
Jackson, P. A., Abd Rahman, W. N. W., Wong, C. J., Ackerly, T. & Geso, M. Potential dependent superiority of gold nanoparticles in comparison to iodinated contrast agents. European Journal of Radiology 75, 104-109 (2010).
Gupta, S., Huda, S., Kilpatrick, P. K. & Velev, O. D. Characterization and optimization of gold nanoparticle-based silver-enhanced immunoassays. Analytical chemistry 79, 3810-3820 (2007).

Although the foregoing subject matter has been described in some detail by way of illustration and example for purposes of clarity of understanding, it will be understood by those skilled in the art that certain changes and modifications can be practiced within the scope of the appended claims.

That which is claimed:

1. A process for preparing one or more magnetic metal oxide particles having a silica or gold-silica nanoshell, the process comprising:
(a) providing a salt solution of a metal;
(b) contacting the salt solution of the metal with a precipitant solution to form a reactant solution;
(c) rapidly micro-mixing the reactant solution to initiate formation of metal oxide crystals under controlled nucleation conditions;
(d) continuing to rapidly micro-mix the reactant solution under high gravity conditions to control crystal growth of one or more metal oxide particles formed therein;
(e) optionally coating the one or more metal oxide particles with a surfactant;
(f) separating the one or more metal oxide particles from the reactant solution and one or more by-products, if present, formed therein;
(g) exposing the one or more coated metal oxide particles to high temperature and high pressure in an inert gas environment for a period of time to form one or more magnetic metal oxide particles; and
(h) coating the one or more magnetic metal oxide particles with silica to form one or more magnetic metal oxide particles having a silica nanoshell.

2. The process of claim 1, further comprising:
(i) amino-terminating the silica coating of the one or more magnetic metal oxide particles having a silica nanoshell;
(j) gold seeding the amino-terminated silica coating of the one or more magnetic metal oxide particles having a silica nanoshell; and
(k) gold plating the gold-seeded one or more magnetic metal oxide particles having a silica nanoshell to form one or more magnetic metal oxide particles having a gold-silica nanoshell.

3. The process of claim 2, further comprising coating the one or more magnetic metal oxide particles having a gold-silica nanoshell with a biocompatible coating.

4. The process of claim 3, further comprising binding a ligand to the biocompatible coating.

5. The process of claim 1, wherein the reactant solution comprises an iron precursor solution comprising anhydrous FeCl₃ and FeCl₂.4H₂O in hydrochloric acid.

6. The process of claim 5, wherein the reactant solution further comprises ammonia.

7. The process of claim 1, wherein the coating comprises citric acid.

8. The process of claim 1, wherein the salt solution comprises a metal salt comprising a metal selected from the group consisting of Fe, Co, Ni, and Sm.

9. The process of claim 8, wherein the metal salt comprises an anionic species selected from the group consisting of chloride, bromide, fluoride, iodide, nitrate (NO₃), sulfate (SO₄), chlorate (ClO₄), and phosphate (PO₄).

10. The process of claim 1, wherein the precipitant solution comprises at least one member selected from the group consisting of NaOH, ammonium hydroxide (NH₄OH), and another hydroxide of Group I or II elements from the Periodic Table of elements.

11. The process of claim 1, wherein the reactant solution comprises at least one member selected from the group consisting of a hydroxide, a carbonate, and a phosphate.

12. The process of claim 1, wherein the surfactant is selected from the group consisting of an organic acid, a lipid, a phospholipid, an oleate, an ester, a sulfate, a diol, and a polymer.

13. The process of claim 1, wherein the exposing of the one or more coated metal oxide particles to high temperature and high pressure is conducted at about 130° C. for about 5 hours.

14. The process of claim 1, wherein the pressure range is from about 1 atmosphere to about 1,000 atmospheres.

15. One or more surfactant-coated magnetic metal oxide particles prepared by the method of claim 1.

16. The one or more surfactant-coated magnetic metal oxide particles of claim 15, wherein the particles have a substantially isotopic shape.

17. The one or more surfactant-coated magnetic metal oxide particles of claim 15, wherein the particles have a dimension ranging from about 30 nm to about 100 nm.

18. The one or more surfactant-coated magnetic metal oxide particles of claim 15, wherein the particles comprise about 76% Fe₃O₄ and about 24% γ-Fe₂O₃.

19. The one or more surfactant-coated magnetic metal oxide particles of claim 15, wherein the particles are substantially free of Fe(OH)₂.

20. A magnetic metal oxide nanoparticle prepared from a high-gravity controlled precipitation reaction, the nanoparticle comprising:
(a) iron oxide crystals having a dimension ranging from about 5 nm to about 100 nm;
(b) optionally a surfactant coating; and
(c) a silica coating;
wherein the nanoparticle has a heating property of greater than about 60 W/g Fe in an alternating current (AC) magnetic field having a frequency of ranging from about 50 kHz and to about 1 MHz and an amplitude ranging from about 0.080 kA/m to about 80 kA/m.

21. The magnetic metal oxide nanoparticle of claim 20, wherein the magnetic metal oxide nanoparticle further comprises a gold coating.

22. The magnetic metal oxide nanoparticle of claim 21, wherein the gold-coated magnetic metal oxide nanoparticle further comprising a biocompatible coating.

23. The magnetic metal oxide nanoparticle of claim 22, wherein the gold-coated magnetic metal oxide nanoparticle comprising a biocompatible coating further comprises a ligand.

24. A biocompatible suspension comprising a magnetic metal oxide nanoparticle of claim 15 and water.

25. A method for treating a diseased tissue, the method comprising:
(a) administering to a tissue or a subject in need of treatment thereof, a therapeutically effective amount of a magnetic nanoparticle having a silica or a gold-silica nanoshell, wherein the magnetic nanoparticle comprises iron oxide crystals prepared from a high-gravity controlled precipitation process; and
(b) subjecting the tissue or subject, or a portion of the tissue or subject to an alternating current (AC) magnetic field having frequency ranging from about 50 kHz to about 1 MHz and having an amplitude (peak-to-peak) ranging from about 0.080 kA/m to about 50 kA/m.

26. The method of claim 25, wherein the diseased tissue comprises a cancer tissue.

27. The method of claim 25, in combination with radiation therapy.

28. The method of claim 25, in combination with radiation imaging.

29. A method of imaging a diseased tissue, the method comprising:
(a) administering to a tissue or a subject in need of treatment thereof, a therapeutically effective amount of a magnetic nanoparticle having a silica or a gold-silica nanoshell, wherein the magnetic nanoparticle comprises iron oxide crystals prepared from a high-gravity controlled precipitation process; and
(b) imaging the magnetic nanoparticle having a silica or a gold-silica nanoshell.

30. The method of claim 29, wherein the imaging is conducted by an imaging technique selected from the group consisting of magnetic resonance imaging, plasmon resonance imaging, x-ray imaging, optical coherence tomography (OCT), and x-ray computed tomography.

31. A magnetic nanoparticle comprising:
(a) a magnetic core comprising an aggregate of at least two magnetic crystalline grains, wherein the aggregate exhibits a collective magnetic phase such that the core has an apparently single magnetic domain phase;
(b) a second magnetic phase or magnetic oxide phase differing from the collective or single domain phase of the core, wherein the second magnetic phase or magnetic oxide phase can intercalate and surround the core; wherein at least one magnetic phase exhibits a high-coercive behavior in a magnetic field and at least one other phase exhibits a low-coercive behavior in a magnetic field relative to the high-coercive magnetic phase;
(c) optionally a surfactant coating; and
(d) a silica coating or a gold-silica coating.

32. The magnetic nanoparticle of claim 31, wherein the core substantially comprises Fe₃O₄ and the second magnetic phase or magnetic oxide phase substantially comprises γ-Fe₂O₃.

33. A kit for treating a diseased tissue, the kit comprising a magnetic metal oxide nanoparticle of claim 15.
